(19) United States
(12) Patent Application Publication
(10) Pub. No.: US 2008/0109070 A1
(43) Pub. Date: May 8, 2008

(54) BIODEGRADABLE ELASTOMERIC SCAFFOLDS CONTAINING MICROINTEGRATED CELLS

(76) Inventors: William R. Wagner, Wexford, PA (US); John Stankus, Campbell, CA (US); Jianjun Guan, Pittsburgh, PA (US); Kazuro Lee Fujimoto, Pittsburgh, PA (US); Alejandro Nieponice, Buenos Aires (AR); Lorenzo Soletti, Pittsburgh, PA (US); David A. Vorp, Pittsburgh, PA (US); Michael S. Sacks, Pittsburgh, PA (US); Todd Courtney, Odenton, MD (US); John E. Mayer, Wellesley, MA (US)

Correspondence Address: JESSE A. HIRSHMAN, ESQ., 1722 MURRAY AVENUE, THIRD FLOOR, PITTSBURGH, PA, 15217, US

(21) Appl. No.: 11/837,235

(22) Filed: Aug. 10, 2007

Related U.S. Application Data
(60) Provisional application No. 60/822,073, filed on Aug. 10, 2006.

(51) Int. Cl. A61F 2/06; A61F 2/08; A61F 2/24

(52) U.S. Cl. 623/1.41; 623/14.11; 623/2.12

(57) ABSTRACT

Described herein are elastomeric materials, and in particular porous biodegradable elastomeric materials which optionally may have microintegrated cells. Also described herein are bioprosthetic devices that can be manufactured using the biodegradable elastomeric materials, non-limiting examples of such devices including pulmonary valves, vocal chords, and blood vessels.

### CROSS REFERENCE TO RELATED APPLICATIONS

This application claims the benefit of U.S. Provisional Patent Application No. 60/822,073, filed Aug. 10, 2006, which is incorporated herein by reference in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant Nos. HL069368 and HL068816 awarded by the National Institutes of Health.

Provided herein are elastomeric materials, and in particular biodegradable elastomeric materials with microintegrated cells. Also provided herein are bioprosthetic devices that can be manufactured using the biodegradable elastomeric materials, non-limiting examples of such devices including pulmonary valves, vocal chords, and blood vessels.

There is a continuing need for the development of suitable materials to repair or to replace biological tissues that are damaged or poorly functioning. In many cases, the outlook for individuals in need of repair or replacement of biological tissues is bleak. For example, it is often difficult to find donors for tissue transplants, and many of the current prostheses that are used in lieu of tissue transplants have significant disadvantages.

Heart valve defects provide one example where the development of suitable materials for treatment of the defects is still needed. For example, each year, 8 out of every 1000 infants are born with a congenital heart defect, affecting a total of about 1,000,000 Americans. Despite the advances in medical technology, anomalies of the pulmonary valve (PV) remain predominant, involving stenosis or atresia of the right ventricular outflow tract. Many of these defects involve replacements of the PV and/or reconstruction of the right ventricular outflow tract (RVOT), with multiple reoperations performed to account for somatic growth. Currently, three types of prosthetic devices are utilized for valve replacement: mechanical, bioprosthetic, and homograft valves. Although valve replacement with these devices generally improves a patient's condition as compared to the case where the valvular heart disease is left untreated, each type of valve replacement device has particular problems. While a mature technology, mechanical valves are thrombogenic and thus require lifelong anticoagulation treatments, which reduces (but does not eliminate) the risk of valve thrombosis and embolization of thrombotic material. These valves are also much more susceptible to infection, and once established, infection is extremely difficult to eradicate without replacing the prosthesis.

Bioprosthetic heart valves continue to have limited durability, due to leaflet mineralization with or without tearing, and mechanical fatigue (such as non-calcific tearing). The majority of degenerated valves have both calcification and leaflet defects, while stenosis due to calcification or mechanical damage alone occur much less frequently. High levels of calcification generally coincides with regions of high flexure or experience localized mechanical forces, such as the commissures and basal attachment. In addition, isolated non-calcific ultrastructural disruption of bovine heart valves has been observed in clinical explants. Cryopreserved homograft valves are thought to contain at least some viable cells, but these “devices” are allografts and can potentially be subjected to immunologic rejection. In general, homograft valves have advantages and disadvantages similar to bovine heart valves, and have additional significant problem of limitations in supply. Moreover, regardless of the design specifics of current prosthetic valve devices, none offers any potential for growth, and therefore pediatric patients requiring valve replacement will require reoperations to place larger devices to accommodate the growth of the patient.

From this one example, it is evident that there is a critical need for new materials that overcomes the disadvantages associated with implants, such as thrombosis, immunologic rejection, limitations in supply, and inability to grow with a patient. While these needs have been illustrated here for bioprosthetic heart valves, similar needs exist for the repair and replacement of other tissues. Accordingly, described herein are new materials and methods that can be used for repairing or replacing damaged or poorly functioning tissue.

### SUMMARY

Described herein are devices, compositions and methods useful in repairing or otherwise treating conditions requiring repair of defective or otherwise deficient tissue. In one embodiment, a prosthetic cardiovascular valve is provided. The cardiovascular valve comprises, for example and without limitation, a leaflet comprising a biodegradable elastomeric scaffold that has anisotropic (having unlike properties in different directions) mechanical properties. In another non-limiting example, the biodegradable elastomeric scaffold is in the form of a non-woven mesh having a plurality of pores. Optionally, cells are microintegrated into the pores of the non-woven mesh.

In another non-limiting embodiment, a method of repairing a damaged venous valve or pulmonary valve is provided. The method comprises implanting in a patient a prosthetic cardiovascular valve as described herein.

In yet another non-limiting embodiment a prosthetic blood vessel comprising a tube comprising a non-woven biodegradable elastomeric scaffold having a plurality of pores. Cells are optionally microintegrated into the pores of the biodegradable elastomeric scaffold.

Also provided is a prosthetic vocal fold. The prosthetic vocal fold comprises a biodegradable elastomeric scaffold, which optionally has microintegrated cells therein.

Thus provided, according to one non-limiting embodiment of the technology described herein, is a prosthetic cardiovascular valve leaflet comprising a biodegradable elastomeric scaffold having anisotropic mechanical properties and comprising cells integrated into the scaffolding. The biodegradable elastomeric scaffold may be a non-woven mesh having a plurality of pores, prepared, for example and without limitation, by electrospraying or electrospinning. The prosthetic cardiovascular valve leaflet may be incorporated into a prosthetic cardiovascular valve in its typical, but not exclusive use. The cells typically are microintegrated into the pores of the non-woven mesh, for example and without limitation, by electrospraying. The cells can be, without limitation, cells chosen from one or more of stem cells, precursor cells, smooth muscle cells, skeletal myoblasts, myocardial cells, endothelial cells, endothelial progenitor cells, bone-marrow derived mesenchymal cells and genetically modified cells. The biodegradable elastomeric scaffold may further comprise a therapeutic agent and/or a growth factor, such as, without limitation, an antiinflammatory agent chosen from one or more of salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, antiinflammatory proteins, and steroidal antiinflammatory agents. The therapeutic agent may be an anticlotting factor, such as, without limitation, heparin. When the therapeutic agent is a growth factor, the growth factor may be chosen from one or more of an angiogenic or neurotrophic factor, basic fibroblast growth factor (bFGF), acidic fibroblast growth factor (aFGF), vascular endothelial growth factor (VEGF), hepatocyte growth factor (HGF), insulin-like growth factors (IGF), transforming growth factor-beta pleiotrophin protein, and midkine protein.

The prosthetic cardiovascular valve leaflet may be adapted to replace a cardiovascular valve leaflet of one of a venous valve, a mitral valve, an aortic valve, a pulmonary valve, and a tricuspid valve, and in certain embodiments, the prosthetic cardiovascular valve leaflet is adapted to replace a cardiovascular valve leaflet of one of a venous valve, a pulmonary valve, and a tricuspid valve.

The biodegradable scaffolding may be any useful scaffolding, such as, without limitation, those described herein, including, without limitation scaffoldings prepared from synthetic or natural polymers, such as those described herein.

Also provided is a method of repairing a damaged pulmonary valve or venous valve in a patient. The method comprises, without limitation, implanting in the patient a prosthetic cardiovascular valve leaflet comprising a biodegradable elastomeric scaffold having anisotropic mechanical properties and comprising cells integrated into the scaffold or a prosthetic cardiovascular valve comprising the prosthetic cardiovascular valve leaflet, as described throughout this document.

In another embodiment, a prosthetic blood vessel is provided. The vessel comprises a tube, wherein the tube comprises a non-woven biodegradable elastomeric scaffold having a plurality of pores, and wherein cells are optionally microintegrated into the pores of the biodegradable elastomeric scaffold. Non-limiting examples of the cells include one or more of stem cells, precursor cells, smooth muscle cells, skeletal myoblasts, myocardial cells, endothelial cells, endothelial progenitor cells, bone-marrow derived mesenchymal cells and genetically modified cells.

In another embodiment, a prosthetic vocal fold is provided. The prosthetic vocal fold comprises a biodegradable elastomeric scaffold as described herein, and wherein cells are optionally microintegrated into the biodegradable elastomeric scaffold. Non-limiting examples of the cells include one or more of stem cells, precursor cells, smooth muscle cells, skeletal myoblasts, myocardial cells, endothelial cells, endothelial progenitor cells, bone-marrow derived mesenchymal cells and genetically modified cells.

### BRIEF DESCRIPTION OF THE DRAWINGS

FIG. 1 shows Trypan blue staining results for SMC viability after various processing treatment (Spraying=SMCs sprayed from spray nozzle, Spray −15 kV=SMCs sprayed from spray nozzle onto −15 kV charged target, Spray −15 kV+e-PEUU=SMCs sprayed from spray nozzle onto −15 kV charged target during PEUU electrospinning. Electrospraying −15 kV=SMCs electrosprayed at 10 kV onto −15 kV charged target, Electrospraying −15 kV+e-PEUU=SMCs electrosprayed at 10 kV onto −15 kV charged target during PEUU electrospinning).

FIGS. 2A-2C show approaches to cellular microintegration. (FIG. 2A) Microintegration using a side-by-side capillary configuration for electrospinning polymer and electrospraying cells onto a large flat target moving on an x-y stage. (FIG. 2B) Microintegration using a perpendicular capillary configuration for electrospinning polymer and electrospraying cells onto a rotating mandrel moving on a linear stage to result in the construct shown in (FIG. 2C).

FIG. 3 provides a schematic of the perfusion bioreactor employed with microintegrated constructs. 13 mm diameter construct discs (a) were placed between O-rings (b) and a support screen (c) of in-line filter holders (d) followed by perfusion at 0.5 mL/min with a multi-channel peristaltic pump (e). Each construct was placed in its own loop consisting of a 32 mL media bag (f), silicon tubing gas exchanger (g) and syringes for media exchange (h).

FIG. 4A shows cell growth in thin SMC microintegrated e-PEUU construct fabricated on a flat target versus TCPS over 1 week in static culture (*p<0.05 increase from 1 day to 1 week). FIGS. 4B and 4C are representative electron micrographs of SMC microintegrated samples from the construct shown in FIG. 4A at 1 week in culture. ((FIG. 4B) scale bar=10 μm, (FIG. 4C) scale bar=100 μm).

FIG. 5A shows initial cellular uniformity in SMC microintegrated e-PEUU fabricated on a mandrel target. FIG. 5B shows Cell growth in thick SMC microintegrated e-PEUU constructs with static versus perfusion culture. Perfusion was initiated after 1 day in static culture. (*p<0.05 increase with perfusion versus static culture).

FIGS. 6A-6H show fluorescent micrographs of SMC microintegrated e-PEUU constructs after one day of static culture (FIG. 6A), day 4 of perfusion culture (FIG. 6B, day 4 of perfusion culture (FIG. 6C), day 7 of perfusion culture (FIG. 6D), day 4 of static culture (FIG. 6E), high cell number surface image of day 4 of static culture (FIG. 6F), day 7 of static culture (FIG. 6G), and high cell number surface image of day 7 of static culture (FIG. 6H). (scale bar=40 μm, red=f-actin and e-PEUU, blue=nuclei).

FIGS. 7A-7F show hematoxylin and eosin stained sections of SMC microintegrated e-PEUU constructs after one day of static culture (FIGS. 7A and 7C), day 4 of static culture (FIGS. 7B and 7E) and day 4 of perfusion culture (FIGS. 7C and 7F). ((FIGS. 7A-7C) scale bar=100 μm, (FIGS. 7D-7F) scale bar=40 μm).

FIGS. 8A-8D show H&E staining or SEM of SMC microintegrated PEUU/collagen (75/25). (FIG. 8A) SMCs are aligned into the plane of the sample after 1 day of static culture. (FIG. 8B) SEM illustrates SMC alignment near the surface of PEUU/collagen after 13 days of perfusion culture (scale bar=10 μm). (FIG. 8C) H&E stain after 13 days of perfusion culture indicating cells aligned into the plane of the image. (FIG. 8D) H&E stain after 13 days of perfusion culture indicating high density cell alignment. Note that perfusion was initiated at 0.5 mL/min after 1 day of static culture.

FIG. 9 is a graph showing MTT data for MDSC microintegrated PEUU after 6 days of culture. Samples were cultured statically for 1 day and then subjected to either perfusion (0.5 mL/min) or static culture for an additional 5 days.

FIG. 10A is a confocal micrograph of MDSC microintegrated PEUU demonstrating high density of aligned cells (red=f-actin, blue=nuclei, scale bar=40 μm). FIG. 10B shows a Masson's Trichrome stained MDSC sample indicating collagen production. Both samples are after 5 days of perfusion culture at 0.5 mL/min.

FIGS. 11A-11C depict microintegrated EPC viability. FIG. 11A shows MTT results after 3 days of static or perfusion culture. FIG. 11B is a confocal micrograph of day 4 static culture sample. FIG. 11C is a confocal micrograph of day 4 perfusion sample.

FIGS. 12A-12G are SEM images of ES-PEUU demonstrating varying degrees of fiber alignment with increased mandrel rotational velocity. (FIG. 12A) Random, (FIG. 12B) 0.3 m/s, (FIG. 12C) 1.5 m/s, (FIG. 12D) 2.5 m/s, (FIG. 12E) 4.5 m/s, (FIG. 12F) 9.0 m/s, (FIG. 12G) 13.8 m/s. FIG. 12H shows overlaid fiber orientation showing the ability of the image analysis algorithm to track fibers, including the avoidance of low contrast regions were the image quality was low.

FIG. 13 shows fiber histograms orientation overlaid on the SEM image from where they were taken, demonstrating a high degree of structural consistency.

FIG. 14 is a graph showing change in fiber alignment as mandrel velocity is increased. The random, 0.3 and 1.5 m/s scaffolds all show very little fiber orientations. An aligned fiber network is evident for mandrel speeds above 2.0 m/s scaffold, with progressively more anisotropy with increasing mandrel velocity.

FIG. 15 provides graphs showing biaxial mechanical results for the preferred and cross-preferred fiber directions. As the mandrel velocity is increased in the preferred fiber direction, the scaffolds become stiffer in the preferred direction due to the higher number of fibers oriented in that direction. The cross-preferred direction witnesses the opposite effect.

FIG. 16A is a schematic of a native pulmonary valve leaflet showing the location of the biaxial test specimen and the circumferential and radial axes. FIG. 16B is a graph showing resulting biaxial data along with the mechanical response of the 13.8 m/s scaffold. Both native tissue and scaffold exhibit a stiff response in one axis (preferred for the PEUU and circumferential for the PV), with an initial compliant response followed by a stiffer response in the other axis (cross-preferred for the PEUU and radial for the PV).

FIG. 17 is a graph showing change in anisotropy index with mandrel velocity, with no change from isotropy (AR=1) at velocities less than ˜2 m/s. At tangential velocities greater than 2 m/s, the AR increased abruptly to ˜1.3, followed by a steady monotonic increase to 1.5 at 14 m/s. These results indicate highly controllable ranges of mechanical anisotropy by adjusting the rotation velocity.

FIGS. 18A and 18B show prediction of the structural model for the effective fiber (FIG. 18A) and fiber orientation for mandrel velocities from 0 to 13.8 m/s (FIG. 18B). Increasing mandrel velocity resulted in both an increase in effective fiber stiffness and fiber alignment.

FIG. 19 is a photograph showing a target used to electrospin 1.3 mm inner diameter porous conduits for blood vessel tissue engineering. The mandrel is rotated at 250 rpm and charged at −3 kV.

FIG. 20 provides images showing the macroscale appearance of electrospun tube. (right=higher magnification).

FIGS. 21A-21C are SEMs of PEUU electrospun conduits. FIG. 21B displays conduit exterior and FIG. 21C displays the conduit cross-section (scale bars=10 μm).

FIG. 22A is a fluorescent micrograph of MDSCs lining the interior of an electrospun tubular conduit. Nuclear (blue, Dapi) and f-actin (green, rhodamine phalloidin) staining indicating cell attachment on polymer lumen (red, autofluorescence) after 24 h of static culture. FIG. 22B is a confocal image stack demonstrating nuclear (blue, draq5) and f-actin (red, rhodamine phalloidin) staining of the PEUU lumen.

FIG. 23 is an image of electrospun vascular graft immediately after implantation to replace a section of a rat aorta.

FIG. 24 shows H&E/Trichrome stains of 2 wk explants of electrospun vascular grafts. Notice the presence of collagenous capsule and neovessels in graft exterior (bottom image) and luminal cell growth (top image).

FIG. 25A is an image of an SMC microintegrated PEUU conduit prepared for insertion into perfusion bioreactor FIG. 25B.

FIGS. 26A and 26B are images showing the gross appearance of SMC microintegrated PEUU tubular constructs after removal from the fabrication mandrel.

FIG. 27 is a graph showing MTT SMC viability data for microintegrated conduits of either PEUU or PEUU/collagen. Perfusion was initiated after 1 day of static culture for cell attachment.

FIG. 28 is a composite photomigrograph showing uniform SMC placement after 1 day of static culture for microintegrated PEUU conduit.

FIG. 29 is a graph showing an averaged stress-strain curve for ring test of SMC microintegrated 4.7 mm electrospun PEUU tube.

FIG. 30 is a graph showing the pressure/diameter relationship comparison between porcine mammary artery (pMA) and SMC microintegrated PEUU tubular constructs (μSMC-PEUU).

FIG. 31 is a schematic of a cross-sectional view of the wall of the urinary bladder (not drawn to scale). The following structures are shown: epithelial cell layer (A), basement membrane (B), tunica propria (C), muscularis mucosa (D), tunica submucosa (E), tunica muscularis extema (F), tunica serosa (G), tunica mucosa (H), and the lumen of the bladder (L).

### DETAILED DESCRIPTION

Described herein are highly cellularized and mechanically functional engineered tissue constructs that are suitable for repairing or replacing tissues such as, for example and without limitation, diseased cardiovascular and other soft tissues. Biodegradable porous scaffolds may be fabricated and concurrently or subsequently seeded with cells, optionally cultured in vitro, and then implanted as a part of a bioprosthetic device. The biodegradable porous scaffolds not only provide mechanical support, but also support cell-cell interactions between the cells that are microintegrated into the scaffolds and direct the alignment of cells to mimic tissue structures. The microintegrated cells induce the growth of new tissue, typically either by proliferation or by expressing substances that induce proliferation of cells surrounding the device. Using appropriate cells for integration, the products and compositions described herein are capable of producing mechanically robust contractile muscle or cardiovascular tissues that consist of high densities of aligned cell morphologies.

By “microintegrated” or “microintegration” it is meant that the cells are integrated into the scaffold on a micron level. As an example, the cells are integrated into the material predominantly as individual cells in contact with the material of the scaffold, or in clusters of cells in the range of up to about 10-25μ(microns) and typically in the range of 1-10μ. By virtue of their intimate contact with the scaffold material into which they are integrated, microintegrated cells are restricted in their ability to wash through or out of the matrix, though they may migrate through the matrix by virtue of their own motility. Microintegration can be achieved thorough electrospraying and electrospinning methods as described herein.

As used herein, the term “polymer” refers to both synthetic polymeric components and biological polymeric components. “Biological polymer(s)” are polymers that can be obtained from biological sources, such as, without limitation, mammalian or vertebrate tissue, as in the case of certain extracellular matrix-derived (ECM-derived) compositions. Biological polymers can be modified by additional processing steps. Polymer(s), in general include, for example and without limitation, mono-polymer(s), copolymer(s), polymeric blend(s), block polymer(s), block copolymer(s), cross-linked polymer(s), non-cross-linked polymer(s), linear-, branched-, comb-, star-, and/or dendrite-shaped polymer(s), where polymer(s) can be formed into any useful form, for example and without limitation, a hydrogel, a porous mesh, a fiber, woven mesh, or non-woven mesh, such as, for example and without limitation, as a non-woven mesh formed by electrospinning.

Generally, the polymeric components suitable for the scaffold described herein may be any polymer that is biodegradable and biocompatible. By “biodegradable”, it is meant that a polymer, once implanted and placed in contact with bodily fluids and/or tissues, will degrade either partially or completely through chemical, biochemical and/or enzymatic processes. Non-limiting examples of such chemical reactions include acid/base reactions, hydrolysis reactions, and enzymatic cleavage.

In certain non-limiting embodiments, the biodegradable polymers may comprise homopolymers, copolymers, and/or polymeric blends comprising, without limitation, one or more of the following monomers: glycolide, lactide, caprolactone, dioxanone, and trimethylene carbonate. In other non-limiting embodiments, the polymer(s) comprise labile chemical moieties, non-limiting examples of which include esters, anhydrides, polyanhydrides, or amides, which can be useful in, for example and without limitation, controlling the degradation rate of the scaffold and/or the release rate of therapeutic agents from the scaffold. Alternately, the polymer(s) may contain peptides or biomacromolecules as building blocks which are susceptible to chemical reactions once placed in situ. In one non-limiting example, the polymer is a polypeptide comprising the amino acid sequence alanine-alanine-lysine, which confers enzymatic lability to the polymer. In another non-limiting embodiment, the polymer composition may comprise a biomacromolecular component derived from an ECM. For example, the polymer composition may comprise the biomacromolecule collagen so that collagenase, which is present in situ, can degrade the collagen.

In some non-limiting embodiments, the polymer is selected so that it degrades in situ on a timescale that is similar to the expected rate of healing of the tissue damage or repair. Non-limiting examples of useful in situ degradation rates include between one week and two years, between two weeks and one year, between one month and six months and increments therebetween. The constituents of the scaffolding and the polymer compounds that make up the scaffolding can be tailored to control in situ degradation rates. Prevalence of labile bonds or structures within the scaffold and their accessibility (whether to enzymatic or chemical degradation), is one parameter that would dictate degradation rates. The nature of the labile bonds or structures within the scaffold also would affect degradation rates. Increases in the number of bonds or structures in the scaffold that are more readily broken in vivo will increase the degradation rate.

By “biocompatible,” it is meant that a polymer composition and its normal degradation in vivo products are cytocompatible and are substantially non-toxic and non-carcinogenic in a patient within useful, practical and/or acceptable tolerances. By “cytocompatible,” it is meant that the polymer can sustain a population of cells and/or the polymer composition, device, and degradation products, thereof are not cytotoxic and/or carcinogenic within useful, practical and/or acceptable tolerances. For example, the polymer when placed in a human epithelial cell culture does not adversely affect the viability, growth, adhesion, and number of cells. In one non-limiting embodiment, the compositions, and/or devices are “biocompatible” to the extent they are acceptable for use in a human veterinary patient according to applicable regulatory standards in a given jurisdiction. In another example the biocompatible polymer, when implanted in a patient, does not cause a substantial adverse reaction or substantial harm to cells and tissues in the body, for instance, the polymer composition or device does not cause necrosis or an infection resulting in harm to tissues from the implanted scaffold.

The mechanical properties of a biodegradable elastomeric scaffold can be optimized to reduce strain and stress on the native tissue at the site of implantation. In certain non-limiting embodiments, the mechanical properties of the scaffold are optimized similar to or identical to that of native soft tissue, such as fascia, connective tissue, blood vessel, muscle, tendon, fat, etc. In one non-limiting embodiment, the biodegradable elastomeric scaffold comprises a thermoplastic elastomeric polymer. The mechanical properties of the scaffold also may be optimized to be suitable for surgical handling. In one non-limiting embodiment, the scaffold is flexible and can be sutured to the site. In another, the scaffold is foldable and can be delivered to the site by minimally invasive laparoscopic methods.

The physical and/or mechanical properties of the biodegradable elastomeric scaffold can be optimized by any useful method. Variables that can be optimized include without limitation, the extent of physical cross-linking in a network comprising polymeric components, the ratio of polymeric components within the network, the distribution of molecular weight of the polymeric components, and the method of processing the polymers. Polymers are typically semicrystalline and their physical properties and/or morphology are dependant upon a large number of factors, including monomer composition, polydispersity, average molecular weight, cross-linking, and melting/crystallization conditions. For example, flow and/or shear conditions during cooling of a polymer melt are known to affect formation of crystalline structures in the composition. In one non-limiting embodiment, the scaffold comprises a polymeric component that provides strength and durability to the scaffold, yet is elastomeric so that the mechanical properties of the scaffold are similar to the native tissue surrounding the wound or site in need of tissue regeneration.

The polymers used to make biodegradable scaffold described herein are typically elastomeric. Generally, any elastomeric polymer that has properties similar to that of the soft tissue to be replaced or repaired is appropriate. For example, in certain embodiments, the polymers used to make the biodegradable elastomeric scaffold are highly distensible. Non-limiting examples of suitable polymers include those that have a breaking strain of from 100% to 1700%, more preferably between 200% and 800%, and even more preferably between 325% and 600%. In certain non-limiting embodiments, the breaking strain of the polymer is between 5% and 50%, between 10% and 40%, or between 20% and 30%, including increments therebetween. Further, it is often useful to select polymers with tensile strengths of from 10 kPa-30 MPa, from 5-25 MPa, or between 8 and 20 MPa, including increments therebetween. In certain embodiments, the initial modulus is between 10 kPa to 100 MPa, between 10 and 90 MPa, or between 20 and 70 MPa, including increments therebetween.

In one non-limiting embodiment, the biodegradable elastomeric scaffold comprises a synthetic polymeric component and a biological polymeric component. The synthetic and biological polymeric components may be selected to impart different properties to the biodegradable elastomeric scaffold. For example and without limitation, the synthetic polymeric component may be selected to provide mechanical strength and durability to the scaffold, as well as certain mechanical properties, as described herein. The biological polymeric component may be a material that encourages tissue regeneration and remodeling within the patient, thereby increasing the rate of wound healing.

The synthetic polymeric component can be any useful biocompatible, biodegradable and elastomeric synthetic polymer material, for example and without limitation as described within this application. In one non-limiting embodiment, the synthetic polymeric component is a polymer that provides durability as assayed in an accelerated fatigue test as described by Bemacca et al. Int J. Artif. Organs, 20(6): 327-331 (1997). In certain non-limiting embodiments, the synthetic polymeric component comprises a thermoplastic biodegradable elastomer. In another the synthetic polymeric component comprises a phase-separated biodegradable elastomer with degradable soft and/or hard segments. In yet another non-limiting embodiment, the synthetic polymeric component comprises any hydrolytically, chemically, biochemically, and/or proteolytically labile group, non-limiting examples of which include an ester moiety, amide moiety, anhydride moiety, specific peptide sequences, and generic peptide sequences.

In certain non-limiting embodiments, the synthetic polymeric component is a biodegradable elastomeric polyurethane polymer. In one example, the synthetic polymeric component is a linear segmented poly(urethane urea) copolymer, where the copolymer comprises alternating blocks of “soft” and “hard” segments. In one non-limiting embodiments, the soft segment is a polyether or polyester (e.g., polycaprolactone), which may have a glass transition temperature (temperature at which a reversible change occurs in an amorphous material, such as glass or an amorphous polymer, or in amorphous portions of a partially crystalline polymer from, or to, a viscous or rubbery condition to a hard or relatively brittle one) below the use temperature. As used herein, the “use temperature” or like phrases refers to the temperature at which the scaffolding is maintained after implantation, namely the body temperature of a patient, such as 37° C. for a human patient.

In another non-limiting embodiment, the soft segment comprises a multiblock copolymer in which one or more segments are polyester. In one non-limiting embodiment, a pre-polymer is formed by reacting butyl diisocyanate with polycaprolactone diol and then further reacting the pre-polymer with a chain extender, such as butyl diamine and specific peptide sequences (e.g., alanine-alanine-lysine).

The synthetic polymeric component can be prepared by any useful method. According to one non-limiting embodiment, the synthetic polymeric component comprises a biodegradable polymeric portion, an isocyanate derivative, and a diamine chain extender. In one non-limiting example, formation of the polymeric component comprises at least two steps. In the first step, a pre-polymer is formed, for example in one non-limiting embodiment, the pre-polymer comprises an isocyanate-terminated polymer, which is formed by reacting a biodegradable polymer with an isocyanate derivative. In the second step, the pre-polymer can be further reacted to form chemical bonds between pre-polymer molecules. For example, the isocyanate-terminated pre-polymer is reacted with a diamine chain extender, which reacts with the isocyanate moiety to form chemical bonds between pre-polymer molecules. In another non-limiting example, the isocyanate-terminated pre-polymer is reacted with a diol chain extender, which reacts with the isocyanate moiety. As used herein, an “isocyanate derivative” is any molecule or group that is terminated by the moiety —N═C═O. Isocyanate derivates also include, without limitation, monoisocyanates and polyisocyanates, such as diisocyanates and triisocyanates. In one non-limiting embodiment, the isocyanate derivative is 1,4-diisocyanatobutane.

Preparation of polymeric components may include other steps, including, for example and without limitation, catalytic steps, purification steps, and separation steps. The synthetic polymeric component described herein comprises one or more biodegradable, biocompatible polymers. The biodegradable polymers may be, without limitation, homopolymers, copolymers, and/or polymeric blends. The polymer(s) may comprise, without limitation, one or more of the following monomers: glycolide, lactide, caprolactone, dioxanone, and trimethylene carbonate. In one non-limiting embodiment, the polymer comprises a polycaprolactone. In another embodiment, the polymer comprises a polycaprolactone diol. In yet another embodiment, the polymer comprises a triblock copolymer comprising polycaprolactone, poly(ethylene glycol), and polycaprolactone blocks.

As used herein, a “chain extender” is any molecule or group that reacts with an active group, such as, without limitation, an isocyanate derivative, to extend chains of polymers. Non-limiting examples of useful chain extenders are diamines and diols. In one non-limiting embodiment, the chain extender is a diamine that allows for extending the chain of the pre-polymer, such as putrescine (1,4-diaminobutane). In another non-limiting embodiment, the diamine is lysine ethyl ester. In yet another non-limiting embodiment, the diamine is a peptide fragment comprising two or more amino acids, for example and without limitation, the peptide fragment alanine-alanine-lysine, which can be cleaved enzymatically by elastase. In one non-limiting embodiment, the chain extender is a diol that allows for extending the chain of the pre-polymer, such as 1,4-butane diol.

In one non-limiting embodiment, the synthetic polymeric component comprises a biodegradable poly(ester urethane) urea elastomer (PEUU). One non-limiting example of a PEUU is an elastomeric polymer made from polycaprolactone diol (MW 2000) and 1,4-diisocyanatobutane, using a diamine chain extender, such as putrescine. The PEUU copolymer can be prepared by a two-step polymerization process whereby polycaprolactone diol (MW 2000), 1,4-diisocyanatobutane, and diamine are combined in a 2:1:1 molar ratio. In the first step, to form the pre-polymer, a 15 wt % solution of 1,4-diisocyanatobutane in DMSO (dimethyl sulfoxide) is stirred continuously with a 25 wt % solution of polycaprolactone diol in DMSO. Then, stannous octoate is added and the mixture is allowed to react at 75° C. for 3 hours. In the second step, the pre-polymer is reacted with a diamine to extend the chain and to form the polymer. For example and without limitation, the diamine putrescine is added drop-wise while stirring and allowed to react at room temperature for 18 hours. In another non-limiting embodiment, the diamine is lysine ethyl ester, which is dissolved in DMSO with triethylamine, added to the pre-polymer solution, and allowed to react at 75° C. for 18 hours. After the two step polymerization process, the polymer solution is precipitated in distilled water. Then, the wet polymer is immersed in isopropanol for three days to remove any unreacted monomers. Finally, the polymer is dried under vacuum at 50° C. for 24 hours.

In another non-limiting embodiment, the synthetic polymeric component comprises a poly(ether ester urethane) urea elastomer (PEEUU). In one non-limiting example, the PEEUU is made by reacting polycaprolactone-b-polyethylene glycol-b-polycaprolactone triblock copolymers with 1,4-diisocyanatobutane and putrescine. PEEUU may be obtained, for example and without limitation, by a two-step reaction using a 2:1:1 reactant stoichiometry of 1,4-diisocyanatobutane:triblock copolymer:putrescine. In a further non-limiting example, the triblock polymer is prepared by reacting poly(ethylene glycol) and ε-caprolactone with stannous octoate at 120° C. for 24 hours under a nitrogen environment. The triblock copolymer may be washed with ethyl ether and hexane, then dried in a vacuum oven at 50° C. In the first step to form the pre-polymer, a 15 wt % solution of 1,4-diisocyanatobutane in DMSO is stirred continuously with a 25 wt % solution of triblock copolymer in DMSO. Stannous octoate is then added and the mixture is allowed to react at 75° C. for 3 hours. In the second step, putrescine is added drop-wise under stirring to the pre-polymer solution and allowed to react at room temperature for 18 hours. The PEEUU polymer solution is then precipitated with distilled water. The wet polymer is immersed in isopropanol for 3 days to remove unreacted monomer and dried under vacuum at 50° C. for 24 hours.

In one non-limiting embodiment, the scaffold comprises a mixture of polymeric components, and at least one component is elastomeric. In that embodiment, the ratio of polymeric components in the mixture can be optimized to obtain an elastomeric mixture of suitable, desirable physical qualities. In one non-limiting embodiment, the mixture has physical properties similar to that of soft tissue such as, without limitation, fascia. In yet another non-limiting embodiment, the mixture comprises at least 90%, 80%, 70%, 60%, 50%, 40%, 30%, 20%, and 10% of the elastomeric polymeric component. For example, according to one embodiment, the mixture comprises 50% of a synthetic polymeric component and 50% of a biological polymeric component, for example and without limitation, the mixture may comprise 50% PEUU by weight and 50% UBM (see below) by weight.

In certain embodiments, the polymers used to make the biodegradable elastomeric scaffold are not only non-toxic and non-carcinogenic, but also release therapeutic agents when they degrade within the patient's body. For example, the individual building blocks of the polymers may be chosen such that the building blocks themselves provide a therapeutic benefit when released in situ through the degradation process. In one embodiment, one of the polymer building blocks is putrescine, which has been implicated as a substance that causes cell growth and cell differentiation.

In another non-limiting embodiment, at least one therapeutic agent is added to the biodegradable elastomeric scaffold before it is implanted in the patient. Generally, the therapeutic agents include any substance that can be coated on, embedded into, absorbed into, adsorbed to, or otherwise attached to or incorporated onto or into the biodegradable elastomeric scaffold that would provide a therapeutic benefit to a patient. Non-limiting examples of such therapeutic agents include antimicrobial agents, growth factors, emollients, retinoids, and topical steroids. Each therapeutic agent may be used alone or in combination with other therapeutic agents. For example and without limitation, a biodegradable elastomeric scaffold comprising neurotrophic agents or cells that express neurotrophic agents may be applied to a wound that is near a critical region of the central nervous system, such as the spine. Alternatively, the therapeutic agent may be blended with the polymer while the polymer is being processed. For example, the therapeutic agent may be dissolved in a solvent (e.g., DMSO) and added to the polymer blend during processing. In another embodiment, the therapeutic agent is mixed with a carrier polymer (e.g., polylactic-glycolic acid microparticles) which is subsequently processed with an elastomeric polymer. By blending the therapeutic agent with a carrier polymer or elastomeric polymer itself, the rate of release of the therapeutic agent may be controlled by the rate of polymer degradation.

In certain non-limiting embodiments, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

In certain non-limiting embodiments, the therapeutic agent is an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazaril, acyclovir, trifluorouridine, foscarnet, penicillin, gentamicin, ganciclovir, iatroconazole, miconazole, Zn-pyrithione, and silver salts such as chloride, bromide, iodide and periodate.

In certain non-limiting embodiments, the therapeutic agent is an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin. Other drugs that may promote wound healing and/or tissue regeneration may also be included.

In certain embodiments, a biological polymer is combined with a synthetic polymer. In one non-limiting embodiment, the biological polymer is provided in the form of an extracellular matrix-derived material. Generally, any type of extracellular matrix (ECM) can be used to prepare the biological, ECM-derived polymeric component of the biodegradable elastomeric scaffold (for example and without limitation, see U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666). By “ECM-derived material” it is meant a composition that is prepared from a natural ECM or from an in vitro source wherein the ECM is produced by cultured cells and comprises one or more polymeric components (constituents) of native ECM.

According to one non-limiting example of the ECM-derived material, ECM is isolated from a vertebrate animal, for example, from a warm blooded mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. The ECM can comprise any portion or tissue obtained from an organ, including, for example and without limitation, submucosa, epithelial basement membrane, tunica propria, etc. In one non-limiting embodiment, the ECM is isolated from urinary bladder, which may or may not include the basement membrane. In another non-limiting embodiment, the ECM includes at least a portion of the basement membrane. In certain non-limiting embodiments, the material that serves as the biological component of the scaffold consists primarily (e.g., greater than 70%, 80%, or 90%) of ECM. In another non-limiting embodiment, the biodegradable elastomeric scaffold may contain at least 50% ECM, at least 60% ECM, at least 70% ECM, and at least 80% ECM. In yet another non-limiting embodiment, the biodegradable elastomeric scaffold comprises at least 10% ECM. The ECM material may or may not retain some of the cellular elements that comprised the original tissue such as capillary endothelial cells or fibrocytes. The type of ECM used in the scaffold can vary depending on the intended cell types to be recruited during wound healing or tissue regeneration, the native tissue architecture of the tissue organ to be replaced, the availability of the tissue source of ECM, or other factors that affect the quality of the final scaffold and the possibility of manufacturing the scaffold. For example and without limitation, the ECM may contain both a basement membrane surface and a non-basement membrane surface, which would be useful for promoting the reconstruction of tissue such as the urinary bladder, esophagus, or blood vessel all of which have a basement membrane and non-basement membrane component.

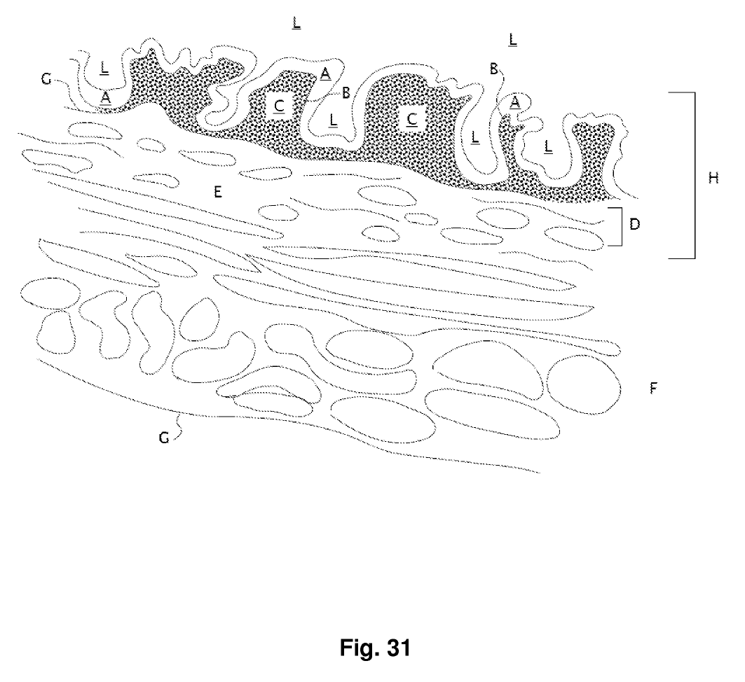

In one non-limiting embodiment, the ECM is harvested from porcine urinary bladders (also known as urinary bladder matrix or UBM). Briefly, the ECM is prepared by removing the urinary bladder tissue from a pig and trimming residual external connective tissues, including adipose tissue. All residual urine is removed by repeated washes with tap water. The tissue is delaminated by first soaking the tissue in a deepithelializing solution, for example and without limitation, hypertonic saline (e.g. 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria (layers B and C of FIG. 31).

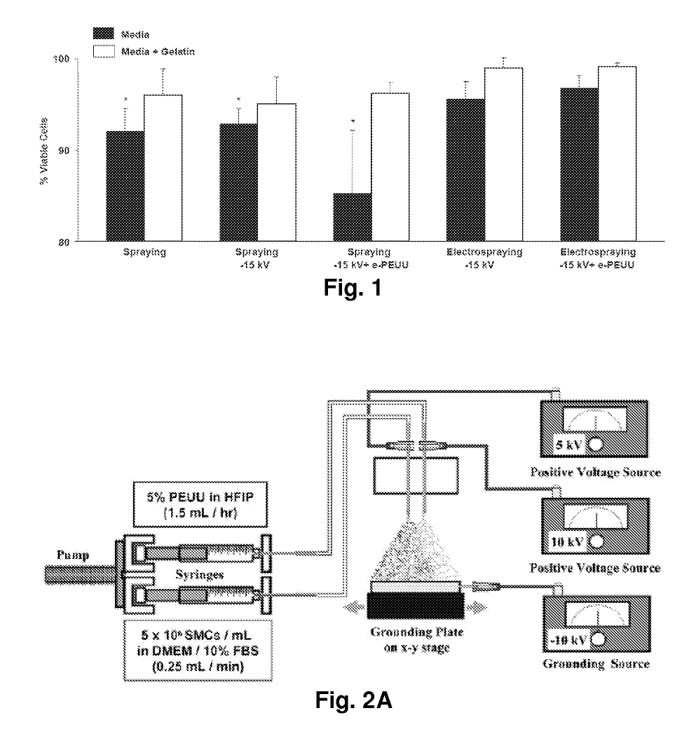

In another embodiment, the ECM is prepared by abrading porcine bladder tissue to remove the outer layers including both the tunica serosa and the tunica muscularis (layers G and F in FIG. 31) using a longitudinal wiping motion with a scalpel handle and moistened gauze. Following eversion of the tissue segment, the luminal portion of the tunica mucosa (layer H in FIG. 1) is delaminated from the underlying tissue using the same wiping motion. Care is taken to prevent perforation of the submucosa (layer E of FIG. 31). After these tissues are removed, the resulting ECM consists mainly of the tunica submucosa (layer E of FIG. 31).

The ECM can be sterilized by any of a number of standard methods without loss of function. For example and without limitation, the material can be sterilized by propylene oxide or ethylene oxide treatment, gamma irradiation treatment (0.05 to 4 mRad), gas plasma sterilization, peracetic acid sterilization, or electron beam treatment. Treatment with glutaraldehyde results in sterilization as well as increased cross-linking of the ECM. This treatment substantially alters the material such that it is slowly resorbed or not resorbed at all and incites a different type of host remodeling, which more closely resembles scar tissue formation or encapsulation rather than constructive remodeling. If desired, cross-linking of the protein material within the ECM can also be induced with, for example and without limitation, carbodiimide isocyanate treatments, dehydrothermal methods, and photooxidation methods. In one non-limiting embodiment, the ECM is disinfected by immersion in 0.1% (v/v) peracetic acid, 4% (v/v) ethanol, and 96% (v/v) sterile water for two hours. The ECM material is then washed twice for 15 minutes with PBS (pH=7.4) and twice for 15 minutes with deionized water. The ECM-derived material may be further processed by optionally drying, desiccation, lyophilization, freeze drying, glassification. The ECM-derived material optionally can be further digested, for example and without limitation by hydration (if dried), acidification, enzymatic digests with, for example and without limitation, trypsin or pepsin and neutralization.

Commercially available ECM preparations can also be used as the biological polymeric component of the scaffold. In one non-limiting embodiment, the ECM is derived from small intestinal submucosa or SIS. Commercially available preparations include, but are not limited to, Surgisis™, Surgisis-ES™, Stratasis™, and Stratasis-ES™ (Cook Urological Inc.; Indianapolis, Ind.) and GraftPatch™ (Organogenesis Inc.; Canton Mass.). In another non-limiting embodiment, the ECM is derived from dermis. Commercially available preparations include, but are not limited to Pelvicol™ (sold as Permacol™ in Europe; Bard, Covington, Ga.), Repliform™ (Microvasive; Boston, Mass.) and Alloderm™ (LifeCell; Branchburg, N.J.). In another embodiment, the ECM is derived from urinary bladder. Commercially available preparations include, but are not limited to UBM (Acell Corporation; Jessup, Md.).

In general, the biodegradable elastomeric scaffold described herein may be made using any useful method, including one to the many common processes known in the polymer and textile arts. The biodegradable elastomeric scaffold may take many different forms. In certain non-limiting embodiments, the biodegradable elastomeric scaffold comprises a thin, flexible fabric that can be sewn directly on to the site to be treated. In another non-limiting embodiment, the scaffold comprises a non-woven mat that can be saturated in place at the site of implantation or affixed using a medically acceptable adhesive. In one non-limiting embodiment, the scaffold is substantially planar (having much greater dimension in two dimensions and a substantially smaller dimension in a third, comparable to bandages, gauze, and other substantially flexible, flat items). In another non-limiting embodiment, the biodegradable elastomeric scaffold comprises a non-woven fibrous article formed by electrospinning a suspension containing the synthetic polymeric component and the biological polymeric component. In yet another non-limiting embodiment, the biodegradable elastomeric scaffold comprises a porous composite formed by thermally induced phase separation.

The biodegradable elastomeric scaffold can also have three-dimensional shapes useful for treating wounds and tissue deficiencies, such as plugs, rings, wires, cylinders, tubes, or disks. A useful range of thickness for the biodegradable elastomeric scaffold is between from about 10 μm (micrometers or microns (μ)) to about 3.5 cm, including increments therebetween, including, without limitation from about 10 μm to about 50 μm, 50 μm to 3.5 cm, 100 μm to 3.0 cm, and between 300 μm and 2.5 cm. In particular embodiments, described herein, the scaffold is formed into one of a tube, to serve as a prosthetic blood vessel, a prosthetic vocal fold, or a cardiovascular valve, such as a venous or pulmonary valve.

In certain non-limiting embodiments, the formation and initial processing of the synthetic polymeric component and the biological polymeric component are separate. For example, the synthesis and dissolution of the synthetic polymeric component may involve solvents that would adversely affect the desirable biological properties of the biological polymeric component. By performing the synthesis and initial processing of the synthetic polymeric component separately from the corresponding synthesis and initial processing steps of the biological polymeric component, it is possible to substantially protect the biological polymeric component against degradation that it would otherwise face when exposed to the solvents used in the synthesis and processing the synthetic polymeric component. In certain non-limiting embodiments, the synthetic polymeric component and biological polymeric component are dispersed in different solvents and subsequently combined (e.g., by combining solvent streams) to form the elastomeric scaffold.

In one non-limiting embodiment, the biodegradable elastomeric scaffold is made by using solvent casting to form a film. This method involves dissolving the polymer in a suitable organic solvent and casting the solution in a mold. For example and without limitation, a 3 wt % solution of the polymer in N,N-dimethylformamide (DMF) is cast into a polytetrafluoroethylene coated dish. Then, DMF typically is evaporated at room temperature and the film is further dried under vacuum.

The biodegradable elastomeric scaffolds may be porous. Porosity may be accomplished by a variety of methods. Although the biodegradable elastomeric scaffolds may be porous or non-porous, it is often advantageous to use a process that produces a porous elastomeric scaffold. Non-limiting examples of such processes include solvent casting/salt leaching, electrospinning, and thermally induced phase separation. In other examples, porosity may be accomplished by creating a mesh of fibers, such as by the aforementioned electrospinning or by ant suitable method of producing a woven or non-woven fiber matrix. As used herein, the term “porosity” refers to a ratio between a volume of all the pores within the polymer composition and a volume of the whole polymer composition. For instance, a polymer composition with a porosity of 85% would have 85% of its volume containing pores and 15% of its volume containing the polymer. In certain non-limiting embodiments, the porosity of the scaffold is at least 60%, 65%, 70%, 75%, 80%, 85%, or 90%, or increments therebetween. In another non-limiting embodiment, the average pore size of the scaffold is between 0.1 and 300μ, 0.1 and 100μ, 1-25μ, including increments therebetween. For example and without limitation, a biodegradable elastomeric scaffold that acts as a barrier to bacteria and other pathogens may have an average pore size of less than 0.5 microns or less than 0.2 microns. When the scaffold is to be manufactured by electrospinning, it is often advantageous to adjust the pore size or degree of porosity by varying the polymer concentration of the electrospinning solution or by varying the spinning distance from the nozzle to the target. For example and without limitation, the average pore size may be increased by increasing the amount of polymeric components within the suspension used for electrospinning, which results in larger fiber diameters and therefore larger pore sizes. In another non-limiting example, the average pore size can be increased by increasing spinning distance from the nozzle to the target, which results in less adherence between fibers and a looser matrix.

The composition of the polymer suspension can affect the physical properties of the resultant elastomeric scaffold. In the biohybrid scaffolding described herein, the synthetic polymeric component typically, but not exclusively, is more mechanically robust than the biological polymeric component. Thus, to produce an elastomeric scaffold with increased mechanical strength, it may be advantageous to increase the amount of synthetic polymeric component relative to the biological polymeric component. On the other hand, to promote rapid healing, it may be advantageous to increase the relative amount of the biological polymeric component if cells grow more readily on the biological polymeric component. In one non-limiting embodiment, PEUU and UBM are mixed at a 1:1 ratio (w/w) and then dissolved at 6 wt % in hexafluoroisopropanol. Nevertheless, the relative ration of biologic and synthetic polymer components may vary greatly from, for example and without limitation, 10,000:1 to 1:10,000 and increments therebetween, including from 1,000:1 to 1:1,000; from 100:1 to 1:100, from 10:1 to 1:10, such as 0.01 wt %, 0.1 wt %, 1 wt %, 2 wt %, 5 wt %, 10 wt %, 25 wt %, 33 wt %, 50 wt %, 67 wt %, 75 wt %, 90 wt %, 95 wt %, 98 wt %, 99 wt %, 99.9 wt % and 99.99 wt % of synthetic polymer as a percentage of the total weight of the synthetic and biological polymeric components.

In certain non-limiting embodiments, the biodegradable elastomeric scaffold is made by using solvent casting and salt leaching. This method involves dissolving the polymeric components that constitute the scaffold into a suitable organic solvent and then casting the solution into a mold containing small particles of predetermined size (known as porogens). Examples of suitable porogens include inorganic salts, crystals of saccharose, gelatin spheres or paraffin spheres. By adjusting the porogen size and/or the ratio of porogen to solvent, the porosity of the final elastomeric scaffold may be adjusted. After casting, the solvent is evaporated, and the resulting polymer composition is immersed into a second solvent that dissolves the porogen, but not the polymer, to produce a porous, sheet-like structure.

In other non-limiting embodiments, electrospinning is used to fabricate the elastomeric scaffold. Electrospinning permits fabrication of scaffolds that resemble the scale and fibrous nature of the native extracellular matrix (ECM). The ECM is composed of fibers, pores, and other surface features at the sub-micron and nanometer size scale. Such features directly impact cellular interactions with synthetic materials such as migration and orientation. Electrospinning also permits fabrication of oriented fibers to result in scaffolds with inherent anisotropy. These aligned scaffolds can influence cellular growth, morphology and ECM production. For example, Xu et al. found smooth muscle cell (SMC) alignment with poly(L-lactide-co-ε-caprolactone) fibers [Xu C. Y., Inai R., Kotaki M., Ramakrishna S., “Aligned biodegradable nanofibrous structure: a potential for blood vessel engineering”, Biomaterials 2004 (25) 877-86.] and Lee et al. submitted aligned non-biodegradable polyurethane to mechanical stimulation and found cells cultured on aligned scaffolds produced more ECM than those on randomly organized scaffolds [Lee C. H., Shin H. J., Cho I. H., Kang Y. M. Kim I. A., Park K. D., Shin, J. W., “Nanofiber alignment and direction of mechanical strain affect the ECM production of human ACL fibroblast”. Biomaterials 2005 (26) 1261-1270].

The process of electrospinning involves placing a polymer-containing fluid (for example, a polymer solution, a polymer suspension, or a polymer melt) in a reservoir equipped with a small orifice, such as a needle or pipette tip and a metering pump. One electrode of a high voltage source is also placed in electrical contact with the polymer-containing fluid or orifice, while the other electrode is placed in electrical contact with a target (typically a collector screen or rotating mandrel). During electrospinning, the polymer-containing fluid is charged by the application of high voltage to the solution or orifice (for example, about 3-15 kV) and then forced through the small orifice by the metering pump that provides steady flow. While the polymer-containing fluid at the orifice normally would have a hemispherical shape due to surface tension, the application of the high voltage causes the otherwise hemispherically shaped polymer-containing fluid at the orifice to elongate to form a conical shape known as a Taylor cone. With sufficiently high voltage applied to the polymer-containing fluid and/or orifice, the repulsive electrostatic force of the charged polymer-containing fluid overcomes the surface tension and a charged jet of fluid is ejected from the tip of the Taylor cone and accelerated towards the target, which typically is biased between −2 to −10 kV. Optionally, a focusing ring with an applied bias (for example, 1-10 kV) can be used to direct the trajectory of the charged jet of polymer-containing fluid. As the charged jet of fluid travels towards the biased target, it undergoes a complicated whipping and bending motion. If the fluid is a polymer solution or suspension, the solvent typically evaporates during mid-flight, leaving behind a polymer fiber on the biased target. If the fluid is a polymer melt, the molten polymer cools and solidifies in mid-flight and is collected as a polymer fiber on the biased target. As the polymer fibers accumulate on the biased target, a non-woven, porous mesh is formed on the biased target.

The properties of the electrospun elastomeric scaffolds can be tailored by varying the electrospinning conditions. For example, when the biased target is relatively close to the orifice, the resulting electrospun mesh tends to contain unevenly thick fibers, such that some areas of the fiber have a “bead-like” appearance. However, as the biased target is moved further away from the orifice, the fibers of the non-woven mesh tend to be more uniform in thickness. Moreover, the biased target can be moved relative to the orifice. In certain non-limiting embodiments, the biased target is moved back and forth in a regular, periodic fashion, such that fibers of the non-woven mesh are substantially parallel to each other. When this is the case, the resulting non-woven mesh may have a higher resistance to strain in the direction parallel to the fibers, compared to the direction perpendicular to the fibers. In other non-limiting embodiments, the biased target is moved randomly relative to the orifice, so that the resistance to strain in the plane of the non-woven mesh is isotropic. The target can also be a rotating mandrel. In this case, the properties of the non-woven mesh may be changed by varying the speed of rotation. The properties of the electrospun elastomeric scaffold may also be varied by changing the magnitude of the voltages applied to the electrospinning system. In one non-limiting embodiment, the electrospinning apparatus includes an orifice biased to 12 kV, a target biased to −7 kV, and a focusing ring biased to 3 kV. Moreover, a useful orifice diameter is 0.047″ (I.D.) and a useful target distance is about 23 cm. Other electrospinning conditions that can be varied include, for example and without limitation, the feed rate of the polymer solutions, the solution concentrations, and the polymer molecular weight.

In certain embodiments, electrospinning is performed using two or more nozzles, wherein each nozzle is a source of a different polymer solution. The nozzles may be biased with different biases or the same bias in order to tailor the physical and chemical properties of the resulting non-woven polymeric mesh. Additionally, many different targets may be used. In addition to a flat, plate-like target, use of a mandrel or a revolving disk as a target is contemplated.

When the electrospinning is to be performed using a polymer suspension, the concentration of the polymeric component in the suspension can also be varied to modify the physical properties of the elastomeric scaffold. For example, when the polymeric component is present at relatively low concentration, the resulting fibers of the electrospun non-woven mesh have a smaller diameter than when the polymeric component is present at relatively high concentration. Without wishing to be limited by theory, it is believed that lower concentration solutions have a lower viscosity, leading to faster flow through the orifice to produce thinner fibers. One skilled in the art can adjust polymer concentrations to obtain fibers of desired characteristics. Useful ranges of concentrations for the polymer component are from 1 wt % to 15 wt %, 4 wt % to 10 wt %, and from 6 wt % to 8 wt %, including increments therebetween for all ranges.

In one non-limiting embodiment, the biodegradable elastomeric scaffold is produced by electrospinning a polymer suspension comprising a synthetic polymeric component and a biological polymeric component. In another non-limiting embodiment, the biodegradable elastomeric scaffold is produced by electrospinning a polymer suspension comprising a synthetic polymeric component from one nozzle and a polymer suspension comprising a biological polymeric component from another nozzle. Non-limiting examples of useful range of high-voltage to be applied to the polymer suspension is from 0.5 to 30 kV, from 5 to 25 kV, and from 10 to 15 kV.

Fabrication and modification of the biodegradable elastomeric scaffold can comprise multiple steps using multiple techniques using polymer compositions that are the same or different. In one non-limiting example, thermally induced phase separation (TIPS) is used to fabricate a portion of the biodegradable elastomeric scaffold and electrospinning may be used to form a fiber coating onto or around the scaffold. In another non-limiting example, solvent casting/salt leaching is used to fabricate a portion of the biodegradable elastomeric scaffold and electrospinning is used to form a fiber coating onto or around the scaffold. The electrospinning solution can contain one or more of any polymeric components, including synthetic polymeric components, biological polymeric components, or mixtures of both. The fiber coating formed by electrospinning can be coated onto or around the entire scaffold or portions of the scaffold.

One or more of therapeutic agents can be introduced into the biodegradable elastomeric scaffold by any useful method, such as, without limitation absorption, adsorption, deposition, admixture with a polymer composition used to manufacture the scaffold and linkage of the agent to a component of the scaffold. In one non-limiting example, the therapeutic agent is introduced into a backbone of a polymer used in the scaffold. By adding the therapeutic agent to the elastomeric polymer itself, the rate of release of the therapeutic agent may be controlled by the rate of polymer degradation. In another non-limiting example, the therapeutic agent is introduced when the scaffold is being made. For instance, during a solvent casting or TIPS process, the therapeutic agent can be added to the solvent with the polymer in the pre-formed mold. During an electrospinning process, the therapeutic agent can be electrosprayed onto the polymer being spun. In yet another non-limiting example, the therapeutic agent is introduced into the scaffold after the patch is made. For instance, the scaffold may be “loaded” with therapeutic agent(s) by using static methods. For instance, the scaffold can be immersed into a solution containing the therapeutic agent permitting the agent to absorb into and/or adsorb onto the scaffold. The scaffold may also be loaded by using dynamic methods. For instance, a solution containing the therapeutic agent can be perfused or electrodeposited into the scaffold. In another instance, a therapeutic agent can be added to the biodegradable elastomeric scaffold before it is implanted in the patient.

Therapeutic agents within the biodegradable elastomeric scaffold can be used in any number of ways. In one non-limiting embodiment, a therapeutic agent is released from the scaffold. For example and without limitation, anti-inflammatory drugs are released from the scaffold to decrease an immune response. In another non-limiting embodiment, a therapeutic agent is intended to substantially remain within the scaffold. For example and without limitation, chemoattractants are maintained within the scaffold to promote cellular migration and/or cellular infiltration into the scaffold.

In one non-limiting embodiment, the biodegradable elastomeric scaffolds release therapeutic agents when the polymeric components degrade within the patient's body. For example and without limitation, the individual building blocks of the polymers may be chosen such that the building blocks themselves provide a therapeutic benefit when released in situ through the degradation process. In one non-limiting embodiment, one of the polymer building blocks is putrescine, which has been implicated as a substance that causes cell growth and cell differentiation.

Cells may be microintegrated with the biodegradable elastomeric scaffold using a variety of methods. For example, the elastomeric scaffold may be submersed in an appropriate growth medium for the cells of interest, and then directly exposed to the cells. The cells are allowed to proliferate on the surface and interstices of the elastomeric scaffold. The elastomeric scaffold is then removed from the growth medium, washed if necessary, and implanted. Alternatively, the cells may be placed in a suitable buffer or liquid growth medium and drawn through the scaffold by using vacuum filtration. But because electrospun non-woven fabrics often have pore sizes that are relatively small (for example, compared to the pore sizes of non-woven fabrics fabricated by other methods such as salt leaching or thermally induced phase separation), culturing cells on the surface of the scaffold or vacuum filtration is usually used when microintegration of cells only near the surface of the elastomeric scaffold is desired.

In another embodiment, the cells of interest are dissolved into an appropriate solution (e.g., a growth medium or buffer) and then sprayed onto a biodegradable elastomeric scaffold while the scaffold is being formed by electrospinning. This method is particularly suitable when a highly cellularized tissue engineered construct is desired. While pressure spraying (that is, spraying cells from a nozzle under pressure) is contemplated herein, in certain non-limiting embodiments, the cells are electrosprayed onto the non-woven mesh during electrospinning. As described herein, electrospraying involves subjecting a cell-containing solution with an appropriate viscosity and concentration to an electric field sufficient to produce a spray of small charged droplets of solution that contain cells. FIG. 1 shows a comparison of cell viability for smooth muscle cells (SMCs) sprayed under different conditions. These different conditions include spraying alone, spraying onto a target charged at −15 kV, spraying onto a target charged at −15 kV with PEUU electro spinning, electro spraying at 10 kV onto a target charged at −15 kV, and electrospraying at 10 kV onto a target charged at −15 kV with PEUU electrospinning. A significant reduction in SMC viability resulted from spraying cells through the nozzle. Without wishing to be bound by theory, it is believed that the physical forces of the pressurized spray in combination with the exposure of cells to processing solvents may have caused this result since viability was lost both from spraying alone and even more so by spraying during electrospun PEUU (e-PEUU) fabrication. Decreased viability from cell aerosol spraying has been reported by others and found to depend largely on nozzle diameter, spray pressure, and solution viscosity [Veazey W. S., Anusavice K. J., Moore K., “Mammalian cell delivery via aerosol deposition”. J. Biomed. Mater. Res. 2005 (72B)334-8.]. Therefore, cells were also sprayed from media supplemented with gelatin to increase viscosity and help protect the cells from mechanical and chemical stresses. Viability was recovered yet the mechanical integrity of the PEUU matrices was disrupted because of gelation within the fiber network.

In contrast to pressurized spraying, electrospraying cells using the methods described herein did not significantly affect cell viability or proliferation. This is consistent with reports by others that cells can survive exposure to high voltage electric fields [see, e.g., Nedovic V. A., Obradovic B., Poncelet D., Goosen M. F. A., Leskosek-Cukalovic O., Bucarski B., “Cell immobiliation by electrostatic droplet generation”, Landbauforsch Volk 2002, (241) 11-17; Temple M. D., Bashari E., Lu J., Zong W. X., Thompson C. B., Pinto N. J., Monohar S. K., King R. C. Y., MacDiarmid A. G., “Electrostatic transportation of living cells through air”, Abstracts of Papers, 223 ACS National Meeting, Orlando, Fla., Apr. 7-11, 2002]. Even in the presence of PEUU electrospinning, SMC viability was not reduced using the methods described herein, perhaps because the positively charged electrospinning and electrospraying streams repelled each other and avoided exposing cells to solvent prior to deposition. Also, due to the relatively large electrospinning distance of 23 cm, PEUU fibers were likely free of solvent by the time they were deposited. Electrospraying from media supplemented with gelatin resulted in a greater number of viable cells compared to electrospraying from media without gelatin. However, the use of gelatin leads to reduced construct mechanical properties. Accordingly, in many cases electrospraying from media alone is the preferred cellular incorporation method.

The cells that may be incorporated on or into the biodegradable scaffold include stem cells, precursor cells, smooth muscle cells, skeletal myoblasts, myocardial cells, endothelial cells, endothelial progenitor cells, bone-marrow derived mesenchymal cells and genetically modified cells. In certain embodiments, the genetically modified cells are capable of expressing a therapeutic substance, such as a growth factor. Examples of suitable growth factors include angiogenic or neurotrophic factor, which optionally may be obtained using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors (IGF), transforming growth factor-beta pleiotrophin protein, midkine protein. In one preferred embodiment, the growth factor is IGF-1.

Processing of Polymers to Form an Elastomeric Scaffold and Uses of the Scaffold

Generally, a biodegradable elastomeric scaffold may be made using processes in the polymer and textile arts. The biodegradable elastomeric scaffold may take many different forms. In certain non-limiting embodiments, the biodegradable elastomeric scaffold is a thin, flexible fabric that can be sewn. For example, when the biodegradable elastomeric scaffold is to be used for a replacement heart valve, a sheet of the scaffold material can be cut to form leaflets that are subsequently attached to a stent (e.g. by sewing or adhesives). The stents may be rigid or slightly flexible and are usually covered with cloth (e.g., a synthetic material such as Dacron™) and attached to a sewing ring for fixation to the patient's native tissue. The leaflet valves described herein may be used to replace any of the heart's four valves. In certain embodiments, the biodegradable elastomeric scaffold has mechanical properties (e.g., mechanical anisotropy) that are similar to that of a native pulmonary valve leaflet, as described herein. In further embodiments, these technologies can be applied to reproduce other cardiovascular valves, such as valves of the venous system, including the pulmonary valve, the tricuspid valve and venous valves, and valves of the arterial system, including the aortic and mitral (bicuspid) valves.

In other embodiments, the biodegradable elastomeric scaffolds may be used to reconstruct or to repair the vocal folds, which are more commonly known as vocal cords. The vocal cords are composed of twin infoldings of a mucous membrane stretched horizontally across the larynx, a cylindrical framework of cartilage that anchors the vocal cords. The vocal cords vibrate when they are closed to obstruct the airflow through the glottis, the space between the folds: they are forced open by increased air pressure in the lungs, and closed again as the air rushes past the folds, lowering the pressure. A person's voice pitch is determined by the resonant frequency of the vocal folds. In an adult male this frequency averages about 125 Hz, adult females around 210, in children the frequency is over 300 Hz.

Provided therefore is a method and compositions for reconstructing damaged vocal cords by surgically implanting a microintegrated biodegradable elastomeric scaffold described herein to provide mechanical reinforcement and/or promote healing. Generally, the biodegradable scaffold is cut in the same general shape as the vocal chords and sewn either directly onto the larynx and/or vocal cords, or onto a supporting ring that is subsequently implanted in the larynx.

In further embodiments, the biodegradable elastomeric scaffold is formed in the shape of a tube (for example, by electrospinning onto a mandrel of appropriate diameter) and used as a prosthesis for hollow organs. For example, a tube-shaped biodegradable elastomeric scaffold may implanted within a patient's body as a prosthetic blood vessel that is fastened to a patient's own blood vessels through the use of surgical fasteners such as sutures or fibrin-based adhesives. In certain embodiments, the tube-shaped scaffold may be formed by removing a smooth muscle cell-integrated scaffold off the mandrel (as a conduit) and seeding the lumen with endothelial cells. The cells may be cultured for a period of, for example, 2-48 hours for the cells to adhere and grow prior to implantation. In other embodiments, precursor or stem cells that might have the potential to turn into vascular cells (SMCs and endothelial cells) may be microintegrated before implantation. In still other embodiments, an additional scaffold is electrospun around the outside of an existing scaffold (seeded or unseeded) to strengthen the mechanical properties. Optionally, cells may be microintegrated during this electrospinning, to create an outer, cellularized layer to the blood vessel.

These same techniques could generally be applied to other conduit structures such as urethra or gastrointestinal structures or sub-structures.

### EXAMPLES

### Example 1

### Microintegration of Smooth Muscle Cells in an Elastomeric Scaffold

This example describes the microintegration of smooth muscles cells in one embodiment of an elastomeric scaffold. The ability to microintegrate smooth muscle cells or other types of cells into a biodegradable elastomericscaffold provides a method for fabricating high density tissue mimetics, blood vessels, leaflets, or other cardiovascular tissues.

1.1 Polymer Synthesis and Characterization

1,4-diisocyanatobutane (BDI, Fluka) and putrescine (Sigma) were distilled under vacuum. Polycaprolactone diol (PCL, MW=2000, Aldrich) was vacuum dried for 48 h. Dimethyl sulfoxide (DMSO) and N,N-dimethylformamide (DMF) were dried over 4-A molecular sieves. Stannous octoate (Sigma) and hexafluoroisopropanol (HFIP, Oakwood Products) were used as obtained.

Cytocompatible and biodegradable PEUU was synthesized from PCL and BDI with subsequent chain extension by putrescine as described herein. The reaction consisted of a two-step solution polymerization in DMSO using a 2:1:1 BDI:PCL:putrescine mole ratio. PEUU cast films were prepared from a 3 wt % solution in DMF and dried under vacuum for 48 h.

The PEUU was characterized for molecular weight, thermal transitions and uniaxial tensile properties. The PEUU number average molecular weight was 88000 and weight average molecular weight was 230,000 as determined by GPC to give a polydispersity of 2.6. DSC values reported a glass transition temperature of −55.0° C. and soft segment melt temperature of 41.0° C. Cast PEUU film was strong and distensible with a tensile strength of 27±4 MPa and a breaking strain of 820±70%.

1.2 Isolation and Culturing of Cells

Vascular SMCs isolated from rat aorta were expanded on tissue culture polystyrene (TCPS) culture plates under Dulbecco's Modified Eagle Medium (DMEM) supplemented with 10% fetal bovine serum and 1% penicillin-streptomycin. SMCs were sprayed from a sterile air pressurized polypropylene bottle with an attached spray nozzle (Fisher) or electrosprayed from a sterile stainless-steel capillary (I.D.=0.047″) at 10 kV over a distance of 20 cm onto glass slides placed on an aluminum plate charged at −15 kV. To shield cells from processing effects and in an effort to maximize viability, some cell suspensions were supplemented with 3 wt % bovine skin gelatin (Sigma) before spraying or electrospraying. For assessment of cell viability, 50 μL of sprayed or electro sprayed SMCs in culture medium were added to 50 μL of 0.4% trypan blue (Gibco). After 5 min incubation, viability was calculated as:

$$\% cell viability = \frac{\# unstained cells ( living )}{\# total cells ( dead + living )} \times 100 \%$$

Murine muscle derived stem cells (MDSCs) were isolated from normal newborn mice through a collagenase based enzymatic digestion method followed by separation based on adhesion characteristics to collagen modified tissue culture flasks (pre-plate method) as described previously. Specifically, MDSCs were clonal colonies of cells that adhered at pre-plate number six. Each pre-plate time consisted of 24 h to allow for cell attachment. These cells have been demonstrated to maintain their phenotype for over thirty subculture periods as well as exhibit the potential to differentiate into muscle, neural, and endothelial cells either in vitro or in vivo. MDSCs were cultured in media that consisted of DMEM supplemented with 10% FBS, 10% horse serum, and 1% penicillin/streptomycin. MSDCs were expanded and microintegrated using the same process variables as described above for SMCs.

Endothelial progenitor cells (EPCs) were isolated from juvenile ovine peripheral blood by a histopaque gradient/pre-plate method and cultured in EBM-2 medium supplemented with EBM-2 SingleQuots without hydrocortisone and 20% fetal bovine serum on 1% gelatin-coated plates. Following 4 to 6 wks expansion and prior to seeding, EPCs were characterized by indirect immunofluorescence as CD31 and vWF positive and a-SMA negative. EPCs were subcultured and microintegrated using identical processing conditions as described above for SMCs.

1.3 Microintegration and Electrospinning

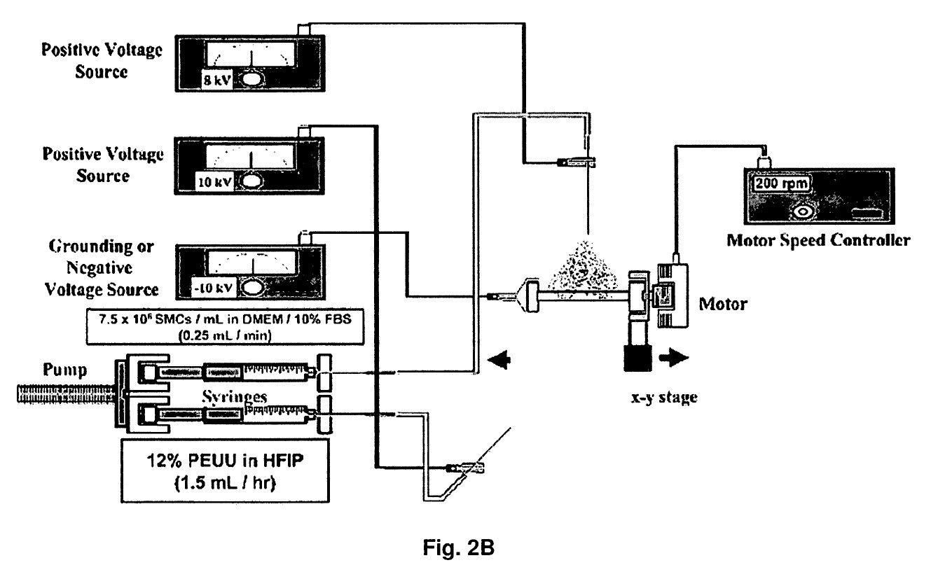

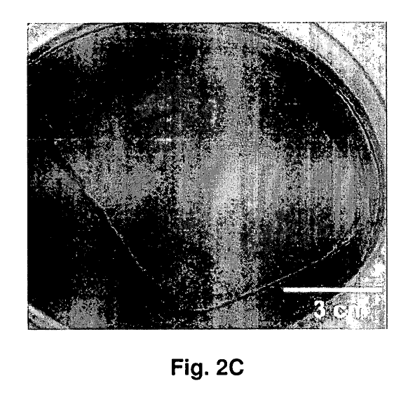

Initial attempts to microintegrate SMCs into electrospun PEUU consisted of side-by-side electrospraying and electro spinning capillaries and a flat conductive target moving on an x-y stage (FIG. 2A)). The capillaries were located 23 cm from the target as depicted in FIG. 2A. 5×10⁶ SMCs/mL in media were fed at 0.25 mL/min with a syringe pump (Harvard Apparatus) through sterile tubing into a sterile capillary charged at 5 kV. PEUU, 5 wt %, was fed at 1.5 mL/hr into a capillary charged at 10 kV. The target was a sterile aluminum plate charged at −10 kV located on an x-y stage (Velmex) translating 8 can along each axis at a speed of 8 cm/s. This technique yielded an approximately 100 μm thick construct after 45 min of fabrication. However, the area of electrospraying and electrospinning stream convergence was relatively small such that non-uniformity of cellular integration was an issue. Without wishing to be bound by theory, it is speculated that this effect was most likely due to a stream repulsion effect from Coulombic forces.

For PEUU/collagen electrospinning, PEUU and type I bovine collagen (Sigma) were dissolved in HFIP under mechanical stirring at a ratio of PEUU/collagen of 75/25 by mass. The polymer solution was fed at 1.5 mL/hr using a syringe pump (Harvard Apparatus PhD) through Teflon tubing and then into a stainless steel capillary (I.D.=0.047″) located 23-cm from a conductive target. High voltage generators (Gamma High Voltage Research) were utilized to charge the polymer solution at 10 kV and the respective target at −10 kV.

In order to fabricate thicker constructs with more uniform cell incorporation, a subsequent microintegration technique was utilized as shown in FIG. 2B. To limit charged stream interactions, the apparatus was modified such that the nozzles were located perpendicular to one another and the target was instead a rotating mandrel translating on its axis (FIG. 2B). Since the electrospun PEUU and electrosprayed SMC streams were arriving from different directions stream repulsion was minimized and the combination of rotation and translation of the mandrel target induced component mixing even further. A total of 7.5×10⁶ SMCs/mL were fed at 0.25 mL/min into a sterile capillary charged at 8 kV and located 5 cm from the target. PEUU. 12 wt %, was fed at 1.5 mL/h into a capillary charged at 10 kV and located 23 cm from the target. The target consisted of a sterile stainless-steel rod (¾″ diameter) charged at 10 kV and rotating at 200 rpm while translating 8 cm along its axis at 8 cm/s. The 5 cm by 5 cm constructs were filleted off the mandrel using a sterile blade by first trimming 1.5 cm off each end before removal. A fabrication time of 45 min was used with both microintegration techniques.

The perpendicular electrospinning/electrospraying nozzles and target configuration may find other applications as a means to fabricate more uniform composite scaffolds by electrospinning multiple materials or introducing drug laden microspheres between fibers. SMC microintegration using this configuration allowed fabrication of approximately 5 cm×5 cm construct sheets of thickness ranging from 300 to 500 μm as shown in FIG. 2C. Scaffold thickness could be controlled by adjusting polymer feed rate or fabrication time. In addition, a more uniform cellular integration was qualitatively visible by observing the overlap of the electrosprayed media and electrospun fibers.

After fabrication, samples were immediately removed from their respective microintegration targets and placed in a sterile polystyrene dish with a minimal amount of culture medium to cover the sample. Areas of the thin SMC mcrointegrated sheets fabricated on the flat target that appeared to possess uniform cell integration with electrospun PEUU were punched into 6-mm discs. These discs were cultured statically in poly 2-hydroxyethyl methacrylate (poly HEMA) coated TCPS 96-well plates with 200 μL of media in each well. As a control, TCPS wells were seeded with SMCs. Media was changed every day.

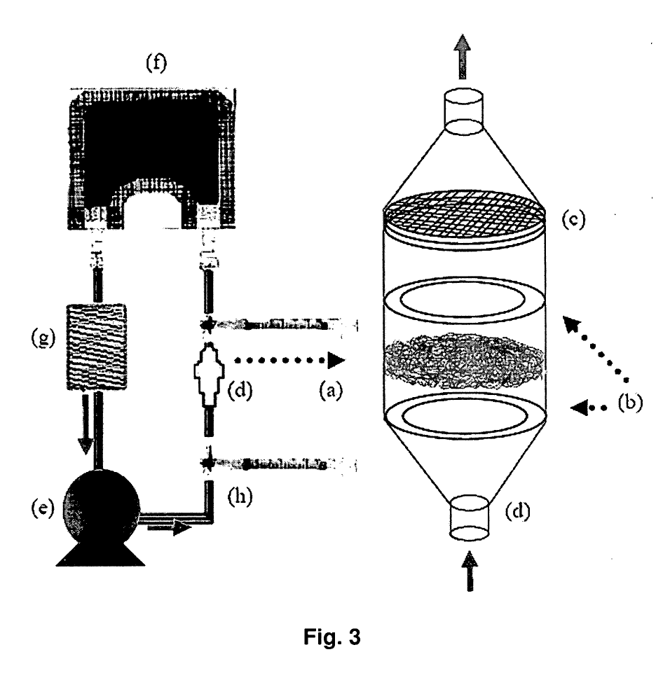

The thicker constructs fabricated using the mandrel target were characterized initially for uniformity of cellular integration. Samples for subsequent study were first cultured with a minimal amount of media to cover the sample for 4 h to encourage cell adhesion. At this point, cells were considered adherent and an additional 15 mL, of media was added to support SMCs for 16 h of static culture. Next, samples were either cultured statically as 6-mm discs in poly HEMA coated TCPS 96-well plates or under transmural perfusion in a custom designed bioreactor. For perfusion culture, samples were cut into 13-mm discs and placed into polypropylene in-line filter holders (VWR) between silicone and Teflon o-rings and a support screen. A schematic of the bioreactor as adapted from a previously reported design is shown in FIG. 3. Each sample was placed in its own flow loop containing a 32-mL media bag (American Fluoroseal Corp), a 2.5 m length of platinum silicone tubing (Cole Parmer, 1/16″ ID.) to serve as a gas exchanger, and two syringes for adding or removing media or bubbles. A multi-channel peristaltic pump (Harvard Apparatus) was utilized to perfuse the loops at 0.5 mL/min. Fifty percent of the media was changed every 2 days.

Quantification of cell viability was achieved using the MTT mitochondrial activity assay (n=5 per sample studied). Regions exposed to flow from samples removed from the bioreactor were punched into 6-mm discs for MTT. For scanning electron microscopy (SEN) to observe cellular and construct morphologies, samples were rinsed with PBS, fixed with 2.5% glutaraldehyde and 1% osmium tetroxide in PBS and subjected to graded ethanol dehydrations before being critical point dried, sputter-coated and imaged. Samples imaged with fluorescence microscopy were rinsed with PBS, fixed with 2% paraformaldehyde, permeabilized with 0.1% Triton x-100 and stained with rhodamine phalloidin (Molecular Probes) for f-actin and draq-5 (Biostatus Ltd) for nuclei. Imaging was done on a Leica TCS-SL laser scanning confocal microscope. Representative images were taken as individual scans or as a series of stacked images. For sectional histology, samples were fixed in 10% neutral buffered formalin, embedded in paraffin, cross sectioned at 10 μm and stained with hematoxylin and eosin. Construct tensile mechanical properties immediately after fabrication using the method shown in FIG. 2B were measured on an ATS 1101 Universal Testing Machine (10 mm/min crosshead speed) according to ASTM D638-98 while wetted with media and immediately after removal from a 37° C. incubator.

1.4 SMC Growth and Morphology

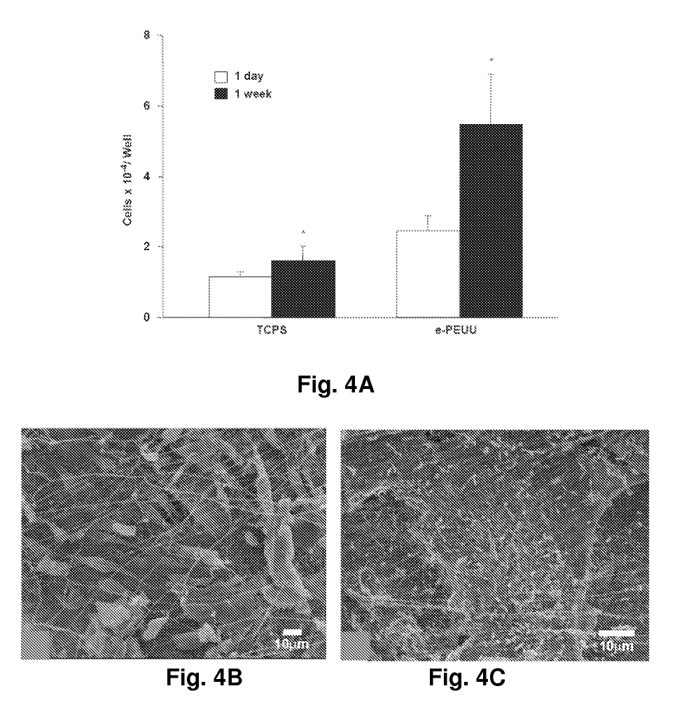

SMC growth in thin constructs fabricated as in FIG. 2A is summarized in FIG. 4A. Cell numbers for both sample types increased significantly from 1 day until 1 week in static culture (p<0.05). SMCs on TCPS increased approximately 40% from 1 day until 1 week while those integrated in electrospun PEUU increased by 122% during this period. Fluorescent imaging of SMC microintegrated PEUU indicated that cells remained spherical in shape at 1 h but exhibited the spread morphology after 1 day of static culture (data not shown). SEM micrographs of fixed samples at 1 week exhibited confluent cellular layers present beneath sub-micron diameter PEUU fibers as shown in FIGS. 4B and 4C.

When thicker SMC microintegrated PEUU scaffolds were submitted to this same static culture method, cells did not proliferate within the construct interior. This effect was attributed to poor exchange of nutrients, waste, and oxygen due to diffusional limitations. Also, cells that followed apoptotic or necrotic pathways remaining in the matrix could detrimentally affect the viability of neighboring healthy cells. Thus, a transmural perfusion bioreactor was constructed to allow increased convective and diffusive transport. This bioreactor was adapted from a report by Radisic et al. who engineered contractile cardiac tissue by exposing neonatal cardiomyocytes seeded into collagen sponges to perfusion culture [Radisic M., Yang L., Boublik J., Cohen R. J., Langer R., Freed L. E., Vunjak-Novakovic G., “Medium perfusion enables engineering of compact and contractile cardiac tissue”., Am. J. Physiol. Heart. Circ. Physiol. 2004 (286)H507-16]. Without washing to be bound by theory, it was hypothesized that that this type of culture system would encourage SMC proliferation in microintegrated constructs and the elastomeric fibers would help retain adherent cells during flow.

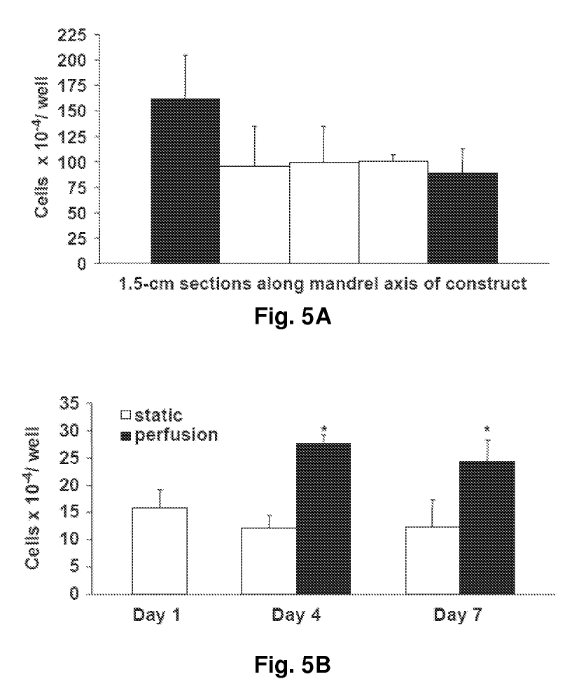

Initial SMC densities in thicker constructs fabricated as in FIGS. 2B and 2C are presented in FIG. 5A. Cell numbers as measured by MTT immediately after construct fabrication ranged from 8.9×10⁴ to 1.6×10⁵ cells/well as a function of position. Although no statistically significant difference was found in cell number with position, constructs were trimmed of 1.5 cm from each edge of the mandrel axis prior to further study. Cellular growth over 1 week with static or perfusion culture is summarized in FIG. 5B. No significant difference in SMC number was found between days 1, 4 or 7 in static culture. However, for samples cultured under transmural perfusion, significantly higher SMC numbers were measured at days 4 and 7 relative to day 1 (p<0.05). These results translate to a 131% and 98% increase in cellular density for perfusion culture versus static culture at days 4 and 7, respectively.

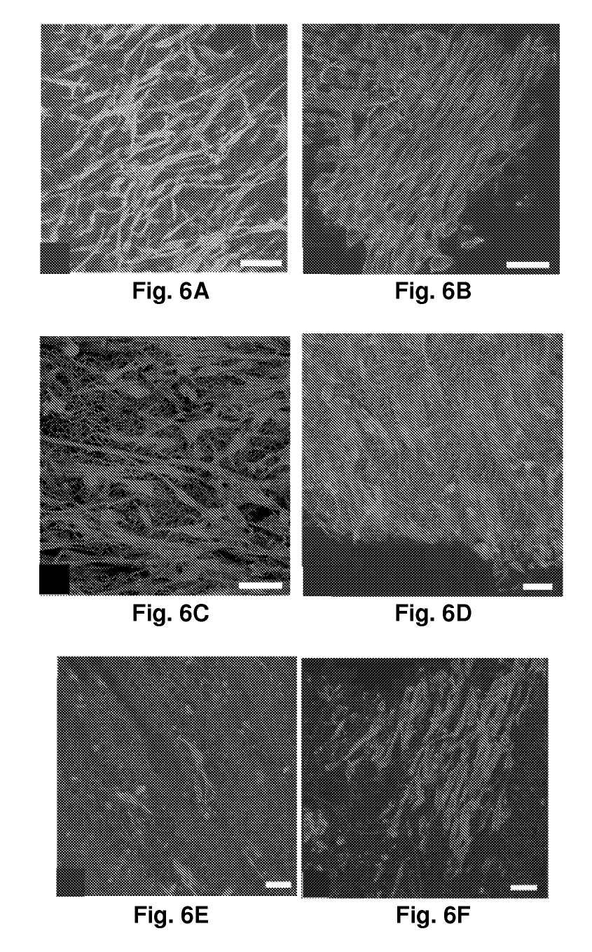

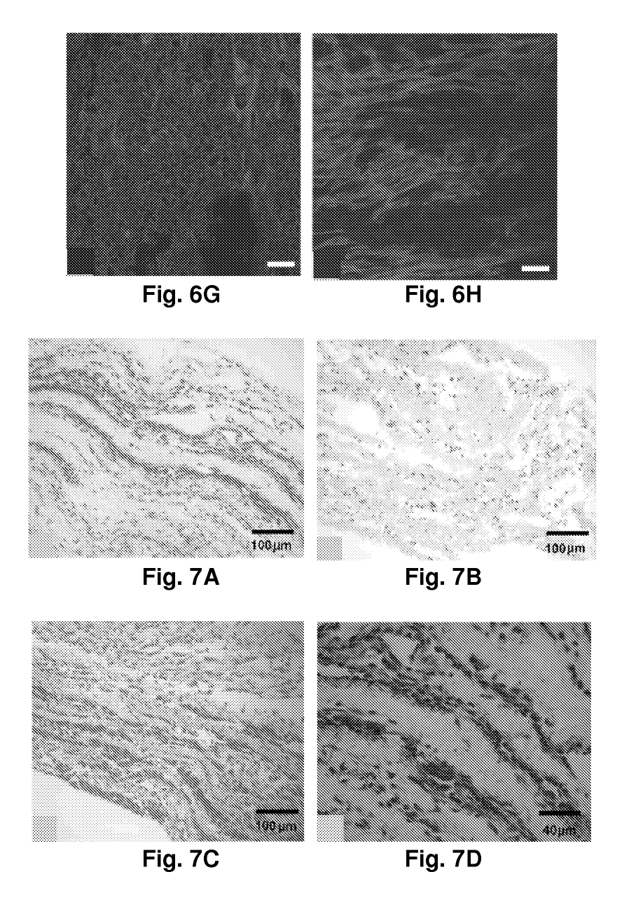

A representative confocal fluorescent image of cellular morphology within the thicker fabricated constructs after 1 day of static culture is shown in FIG. 6A. SMCs appeared spread and healthy as well as uniformly distributed within the scaffold. In addition, constructs cultured under perfusion exhibited high numbers of spread, healthy appearing cells uniformly located throughout the samples as demonstrated in representative images shown in FIGS. 6B-6D. With perfusion, SMCs were found distributed in greater abundance throughout the fiber matrix as well as deeper beneath the fibers. However, at days 4 and 7 of static culture, as displayed representatively in FIGS. 6E and 6G, the SMCs appeared less abundant as well as exhibited less f-actin staining. Patches of higher cell densities were found at both days 4 and 7 of static culture near the construct surface and not deeper in the fiber network as shown in FIGS. 6F and 6H. The morphology of SMCs at day 7 of static culture did improve slightly in appearance in comparison with day 4.

Hematoxylin and eosin stains of construct cross-sections in FIGS. 7A-7F further illustrated the trend of higher cellular density achieved with perfusion culture. One can observe high numbers of layered cells after 1 day of static culture in FIGS. 7A and 7D. Yet, after 4 days of static culture, the cells appear less spread and healthy in FIGS. 7B and 7E. High densities of SMCs microintegrated within the elastomeric fiber network can be observed in FIGS. 7C and 7F after 4 days of perfusion culture.

As a result of the electrospinning setup that was used, it was possible to induce fiber orientation to influence the cells to organize themselves in an aligned manner. SMCs within the elastomeric fiber matrices qualitatively exhibited an aligned morphology, as seen in FIG. 6B for instance. The estimated shear stress to which the SMCs integrated into e-PEUU matrices (at approximately 80% porosity) were exposed in perfusion culture was on the order of 1 dyne/cm². This shear stress is relatively low and would not be expected to significantly influence cell morphology or decrease viability. Additionally, SMC orientation was observed to be parallel to the direction of scaffold fiber orientation instead of aligned with the perfusion flow direction.

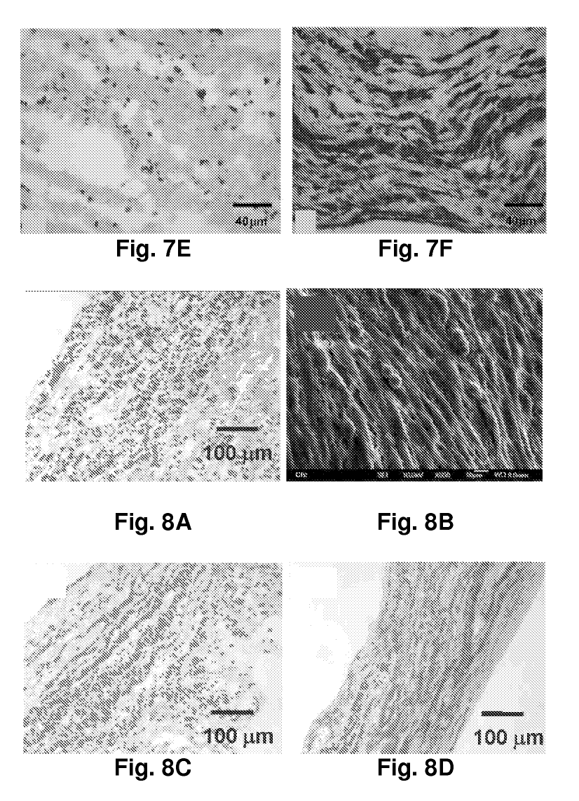

Cell alignment seemed even more qualitatively pronounced in the SMC microintegrated PEUU/collagen (75/25) samples. One can observe the high numbers of microintegrated cells after 1 days of static culture in FIG. 5A. The cells are in high density but do not appear very spread or elongated. This may be due to cells aligning themselves into the plane of the image. For example, when observing the SMCs integrated into electrospun PEUU/collagen after 13 days of perfusion culture (0.5 mL/min) one can observe the same cell morphology in FIG. 8B. However, whenever the sample is sectioned along its other axis (preferred direction of fiber alignment), one can observe high numbers of elongated SMCs aligned with this material axis in FIG. 5D. Also, near the surface of the SMC microintegrated PEUU/collagen are aligned at 14 days after fabrication as well (FIG. 8B).

1.5 MDSC Microintegration, Culture, and Characterization

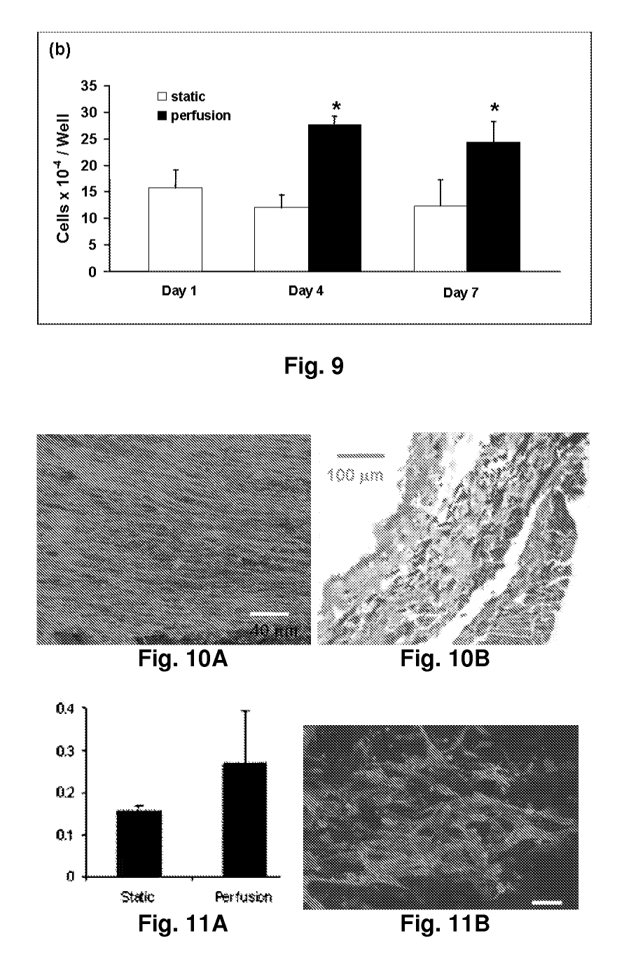

Using identical processing conditions as described above for SMC microintegration, MDSCs were microintegrated into electrospun PEUU at high density. These constructs were also mechanically thick and robust with an almost identical appearance to SMC integrated constructs. These MDSC samples were also subjected to one day of static culture and then 5 days of perfusion culture. MTT data indicated viable cells present 1 day after fabrication (FIG. 9). Significantly higher cell numbers were present at day 3 and day 6 after fabrication with both static and perfusion samples compared with day 1 (p<0.05). These values were different from the trend seen with static culture of SMC microintegrated constructs that did not increase in cell number after 1 day. These results may have been due to the more highly proliferative nature of the MDSCs. In addition, significantly higher cell numbers were observed with perfusion culture in comparison to static culture. This trend was consistent with that observed with SMC culture under perfusion. MTT results were summarized in FIG. 9.

Confocal micrographs taken after 5 days of perfusion culture indicated a high density of aligned cells within the MDSC microintegrated construct (FIG. 10A). These samples appeared even higher in cell density than the SMC microintegrated confocal micrographs after 6 days of perfusion culture. This image together with the relative values for cell numbers from the MTT data for both MDSC and SMC micro integrated constructs generally indicated a higher proliferative capacity for the MDSCs. Masson's Trichrome stained samples from 5 days of MDSC perfusion culture indicated production within the elastomeric PEUU fiber network (FIG. 10B).

1.6 EPC Microintegration, Culture, and Characterization

Using identical processing conditions as described above for SMC microintegration, EPCs were microintegrated into electro spun PEUU. These constructs were also mechanically robust and possessed a similar appearance to SMC integrated constructs. These EPC samples were subjected to one day of static culture and then 3 days of perfusion culture at 0.5 mL/min. MTT data indicated viable cells present for both static and perfusion culture 4 days after fabrication (FIG. 11A). Spread EPCs were observed in confocal micrographs after 4 days of static culture and perfusion culture (FIGS. 11B and 11C. However, FIG. 11C is representative of higher numbers of cells located deeper within the EPC microintegrated fiber networks after perfusion.

1.7 Mechanical Properties

Tensile mechanical properties of SMC microintegrated PEUU measured immediately after fabrication are summarized in Table 1 and compared with e-PEUU. e-PEUU was found to retain much of the mechanical strength and flexibility of the cast film (reported above). SMC microintegrated PEUU was found to retain a portion of the mechanical strength and distensibility of e-PEUU, with lower tensile strengths and higher breaking strains. This latter result may be due to microintegrated SMCs disrupting the PEUU fiber network and replacing elastic PEUU volume with cellular volume. Yet, the measured properties are still more than sufficient for the SMC microintegrated PEUU to serve as a support structure for soft tissue growth and mechanical training.

TABLE 1
Tensile properties of SMC microintegrated PEUU

| | Initial | 100% | Tensile | |
|---|---|---|---|---|
| | modulus | modulus | strength | Breaking |
| Sample | (MPa) | (MPa) | (MPa) | Strain (%) |
| e-PEUU (random) | 2.5 ± 1.2 | 2.8 ± 1.1 | 8.5 ± 1.8 | 280 ± 40 |
| μSMC-e-PEUU | 1.7 ± 0.2 | 1.4 ± 0.2 | 6.5 ± 1.6 | 850 ± 200 |
| (preferred) | | | | |
| μSMC-e-PEUU | — | 0.3 ± 0.1 | 2.0 ± 0.5 | 1700 ± 100 |
| (cross-preferred) | | | | |
| μSMC-e- | | | 3.9 ± 0.9 | 160 ± 40 |
| PEUU/collagen | | | | |
| (75/25) (preferred) | | | | |
| μSMC-e- | | | 0.7 ± 0.1 | 170 ± 40 |
| PEUU/collagen | | | | |
| (75/25) (cross-prefer) | | | | |

E = electrospun scaffold;
μSMC = SMC microintegrated

As a result of the fabrication process, SMC microintegrated PEUU was found to have tensile properties that differed as a function of the material axis. The axis orientated with the mandrel axis (preferred axis) possessed a significantly higher tensile strength and 100% modulus and a lower breaking strain than the axis orientated with the circumference of the mandrel (cross-preferred axis) (p<0.05). Some degree of fiber alignment in the matrices was induced by a combination of the stage translation speed of 8 cm/s and the mandrel length to diameter ratio of 8. Without wishing to be bound by theory, it was believed that this ratio provided more opportunity for the fibers to deposit parallel to the mandrel axis. Since the mandrel rotation velocity was less (3 cm/s at 200 rpm) than the translation speed, it was not expected to greatly influence fiber alignment. As would be expected, the preferred fiber axis possessed a higher tensile strength and lower breaking strain from a more direct influence on the stretching of the fibrous microstructure of the PEUU. The cross-preferred material axis would be expected to allow more elongation at lower stresses since the mechanical properties would be more influenced by PEUU fiber bending than stretching. By manipulating mandrel rotation and translation rates it should be possible to alter the direction and degree of construct anisotropy. This inherent construct anisotropy and fiber orientation appeared to induce the previously mentioned SMC alignment within the matrices.

### Example 2

### Construction of an Anisotropic Elastomeric Material for Pulmonary Heart Valve Leaflet Reconstruction

This example discusses the fabrication of an anisotropic elastomeric material suitable for pulmonary heart valve reconstruction. The example also provides a structural constitutive model that can be used to predict a priori the mechanical properties of non-woven scaffolds from the properties and arrangement of their constituent fibers.

2.1. PEUU Synthesis

Cytocompatible poly(ester urethane) urea (PEUU) was synthesized from polycaprolactone diol and 1,4-diisocyanatobutane with subsequent chain extension by putrescine as described herein. PEUU transparent films were cast from a 3-wt % solution in DMF and dried under vacuum for 48 h. Polymer molecular weight was determined by gel permeation chromatography with 1-methyl-2-pyrrolidione as solvent. Differential scanning calorimetry (Shimadzu DSC 60) was run under helium purge at a scan rate of 20° C./min from −100 to 250° C.

2.2. Electrospun PEUU Fabrication

By syringe pump into a stainless-steel capillary suspended 13-cm vertically over a 4.5″ diameter aluminum mandrel 5-wt % PEUU solution in hexafluoroisopropanol (HFIP) was fed at 1.0 mL/h. PEUU was charged with +12 kV and the aluminum target with −7 kV using high voltage generators (Gamma High Voltage Research). Aligned PEUU fibers were formed by electrospinning onto the target rotating at speeds ranging from 0.0 to 13.8 m/s. Scaffolds were allowed to dry overnight at room temperature and then placed under vacuum for 48 h at 30° C. A portion of each sample was mounted into a standard X-ray diffraction holder for analysis so that the fiber orientation was parallel to the X-ray beam. The samples were run on a PANalytical X'Pert Pro diffractometer using copper radiation. PEUU number average and weight average molecular weight were 228,700 and 87,600, respectively, resulting in a polydispersity index of 2.61. DSC demonstrated a glass transition temperature of −54.6° C. and a melt temperature of the PEUU soft segment at 41.0° C.

2.3. Image Acquisition and Structural Characterization

ES-PEUU samples (10-mm²) were sputter coated with Pd/Au and imaged (grayscale, 8-bit) with scanning electron microscopy (SEM, 1EOL 1SM6330F) to characterize fiber morphologies. The samples were excised from intact ES-PEUU with the known preferred orientation of the polymer parallel to the y-axis of the device. The samples were imaged at 3500× magnification, with each image measuring 1280×1024 pixels and an average image area of ˜1000 μm². Six images were taken from random locations of each sample to minimize local orientation effects.

To quantify the fiber alignment from the scanning electron micrograph (SEM) images, custom image analysis software was developed. Fiber orientation was determined using an algorithm developed by Chaudhuri [Chaudhuri B. B., Kundu P., Sarkar N., “Detection and gradation of oriented texture”, Pattern Recogn. Lett. 1993 14(2): 147-53], modified by Karlon [Karlon W. J., et al., “Automated measurement of myofiber disarray in transgenic mice with ventricular expression of ras”, Anat. Rec. 1998, 252(4): 612-25] and written in MATLAB software (The MathWorks). The vertical and horizontal masks were 7×7 pixels (s=3) with σ=2.5. Sub-regions were chosen based on background color and fiber size. The average background color of the image was determined by choosing pixels in two regions representing dark areas or the background color of the image. If the center pixel of the sub-region and the four pixels adjacent to the center were equal to or less than the background color, that region was omitted during calculations. This allowed the code to skip regions of low gradient change where the fiber tracking algorithm was not effective. The pixel size of the sub-region was chosen based on fiber diameter. Using a MATLAB script, the diameters of 6 different fibers were measured and the average diameter size of the fibers was used as the pixel size of the sub-region. Karlon et al. used all pixels within the sub-region in the weighted accumulator function. The algorithm employed herein used 7 rows of 7 columns, evenly spaced, for a total of 49 pixels. These were input into the accumulator function and, using a range of 0°-179° representing the range of possible orientations, the summed gradient-weighted contribution of each pixel was calculated for each angle. The maximum accumulator bin value was chosen as the dominant orientation within that sub-region. The data from the entire image were then placed into a histogram. The histogram data from each image of a sample were averaged, with the result being the orientation data for the structural model.

2.4. Biaxial Mechanical Testing

The biaxial testing procedure used here has previously been described [Sacks M. S., “Biaxial mechanical evaluation of planar biological materials”, J. Elasticity 2000 (61): 199-246]. In the present study 20×20 mm specimens were used, with the specimen edges aligned to the longitudinal and circumferential axes of the mandrel. A tissue marking dye (Cancer Diagnostics) was used to form four small markers (˜1 mm diameter) in the central 4×4 mm region of the specimen used to compute local strains using an established method [Sacks M. S., “Biaxial mechanical evaluation of planar biological materials”, J. Elasticity 2000 (61): 199-246]. The resulting deformation gradient tensor F was computed, from which the axial stretches λ_{PD}=F₁₁, and λ_{XD}=F₂₂ were determined, where PD and XD refer to the preferred and cross-preferred fiber directions, respectively. All testing was performed in water at room temperature. During all tests the maximum Lagrangian membrane tension tensor T (force/original unit length) level was chosen as 90 N/m. Membrane tension was chosen for run-time control to facilitate comparisons to previous studies on valvular tissues [Billiar K. L., et al, “Biaxial mechanical properties of the natural and gluteraldehyde treated aortic cusp—Part I: experimental results”, J. Biomech. Eng. 2000:122(1):23-30; Grashow J. S. et al., “Biaxial mechanical behavior of the mitral valve anterior leaflet at physiologic strain rates”, Ann. Biomed. Eng., in press]. For constitutive modeling, membrane tensions were converted to the Lagrangian stress tensor P (force/original cross-sectional area). All test protocols maintained a constant ratio of membrane tension (T_{PD}:T_{XD}) throughout cycling. Testing began with two equi-biaxial protocols of T_{PD}:T_{XD} equal to 90:90 N/m. The next 7 consecutive tests were performed with T_{PD}:T_{XD} equal to 9:90, 45:90, 67.5:90, 90:90, 90:67.5, 90:45, and 90:9, respectively. These ratios were chosen to cover a wide range of stress states. A final equi-biaxial test was performed to determine if mechanical behavior changed during the experiment. Total testing time was approximately 2 h per specimen. We calculated the anisotropy ratio (AR) using AR=(λ_{XD}−1)/((λ_{PD}−1), representing the amount of mechanical anisotropy of the specimens.

2.5. Scaffold Structural Constitutive Model

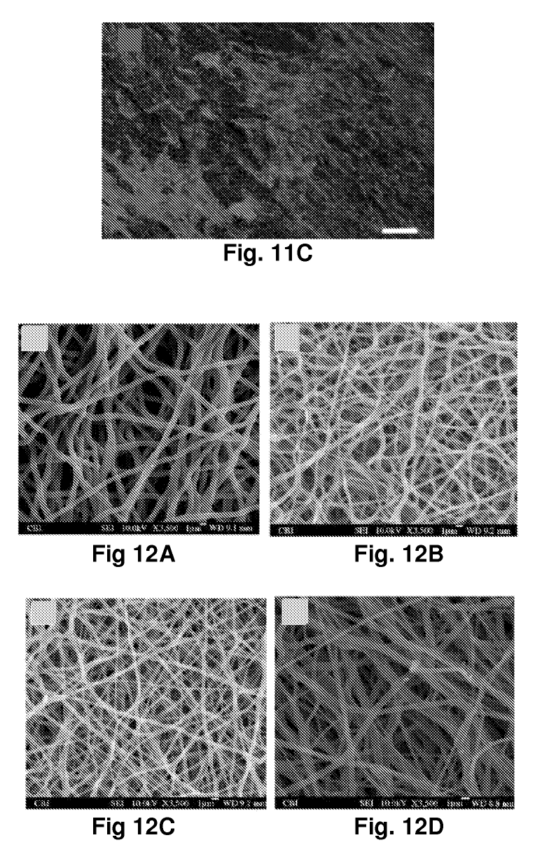

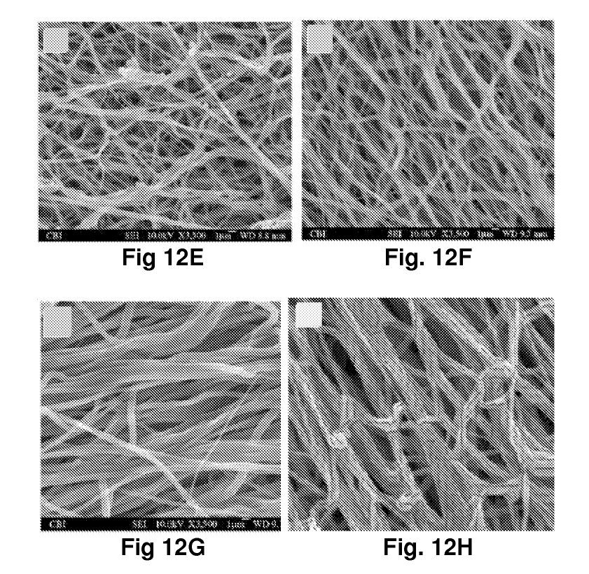

In order to quantitatively relate the angular distribution of ES-PEUU fibers to the resulting planar biaxial mechanical response, a structural approach for constitutive modeling of planar tissues was applied to model the ES-PEUU mechanical response. In this approach, it is assumed that a representative volume element (RVE) can be identified that is large enough to represent the microstructure of the material in some average sense, yet small compared to the characteristic length scale of the microstructure, that is, the thickness. The RVE is treated as a three dimensional continuum and it is assumed that the material can be modeled as a hyperelastic solid. Within the RVE, the following assumptions are made:
1. ES-PEUU can be idealized as a planar network of fibers. Further, since there is no tissue fluid to consider, hydrostatic forces generated that are normally present in tissue do not exist.
2. The ES-PEUU fibers are undulated, which gradually disappears with stretch. The load required to straighten the fiber is considered negligible compared to the load transmitted by the stretched fibers. Hence, fibers transmit load only if stretched beyond the point where all the undulations have disappeared.
3. The fiber strain can be computed from the tensorial transformation of the global strain tensor referenced to fiber coordinates (i.e. the affine transformation assumptions). This is justified from the large number of fiber interconnections (FIGS. 12A-12H).
4. The strain energy function of the scaffold is the sum of the individual fiber strain energies.

To simulate the effective fiber stress-strain law, the simplest formulation (including the fewest number of parameters) was desired, which incorporated the effects of fiber volume fraction, uncrimping, and the intrinsic properties of fiber. For this approach, an exponential form was used:
S_{f}(E_{f})=A[exp(B[E_{f}(θ)])−1
where

$${E}_{f} ( θ ) = \frac{1}{2} ( {λ}_{f}^{2} - 1 ) , \\ {λ}_{f}^{2} = ( {F}_{11}^{2} + {F}_{21}^{2} ) {cos ( θ )}^{2} + 2 ( {F}_{11} {F}_{12} + {F}_{22} {F}_{21} ) cos ( θ ) sin ( θ ) + ( {F}_{22}^{2} + {F}_{12}^{2} ) {sin ( θ )}^{2} , \qquad ( 1 )$$

where A and B are positive constants, λ_{f} and E_{f} represent the fiber stretch and Green's strain, respectively, and Fᵢⱼ are the components of the deformation gradient tensor determined from the biaxial test.

Based on assumption 4, the total scaffold strain energy W can be expressed as:

$$W = {\int }_{- π / 2}^{π / 2} R ( θ ) w [ {E}_{f} ( θ ) ] ⅆ θ , \qquad ( 2 )$$

where w is the fiber strain energy function and R(θ) represents the ES-PEUU fiber orientation distribution, subjected to a normalization constraint:

$${\int }_{- π / 2}^{π / 2} R ( θ ) ⅆ θ = 1.$$

Based on the experimental orientation data, the fiber orientation statistical distribution function R(θ) was modeled using the following modified Cauchy distribution:

$$R ( θ ) = \frac{d}{π} + {( 1 - d ) [ π c [ 1 + {( \frac{x - L}{c} )}^{2} ] ]}^{- 1} \qquad ( 3 )$$

where d represents the random orientation component, and c and L are the shape and location parameters, respectively. Note that L=0 since the specimens are aligned to x-axis. Also, as d→1, the contribution from the random component increases and that from the original Cauchy distribution vanishes. The resulting expressions for the Langrangian stress tensor P are:

$${P}_{11} = {\int }_{- π / 2}^{π / 2} {S}_{f} [ {E}_{f} ( θ ) ] R ( θ ) ( {F}_{11} {cos}^{2} θ + {F}_{21} sin θcos θ ) ⅆ θ , \\ {P}_{22} = {\int }_{- π / 2}^{π / 2} {S}_{f} [ {E}_{f} ( θ ) ] R ( θ ) ( {F}_{22} {sin}^{2} θ + {F}_{12} sin θcos θ ) ⅆ θ , \qquad ( 4 )$$

The material parameters A, B, c, and d were estimated by fitting Eqs. (4) to the complete biaxial data set. Eqs. (4) were solved numerically using Romberg integration, with S_{f}[E_{f}(θ)] set to zero when E_{f}≦0 since fibers cannot support compressive stresses. Finally, while the data was fit using Eqs. (4), this study presents the data in terms of membrane tension T to facilitate comparisons to heart valve tissues.
2.6 Structural Analysis

Electrospinning the polymer solution onto a stationary or rotating mandrel at varying velocities yielded scaffolds that exhibited both structurally isotropic and highly anisotropic fiber networks (FIG. 12A-12G). The random (flat sheet) specimens and those electrospun onto a mandrel with low tangential velocities (in the range of 0.3-1.5 m/s) exhibited fairly isotropic networks, with no discernible difference between the flat sheet and the 1.5 mls scaffolds. Aligned fiber networks developed when the mandrel velocity equaled 3.0 m/s or greater, with a very noticeable increase in alignment as the mandrel velocity was further increased.

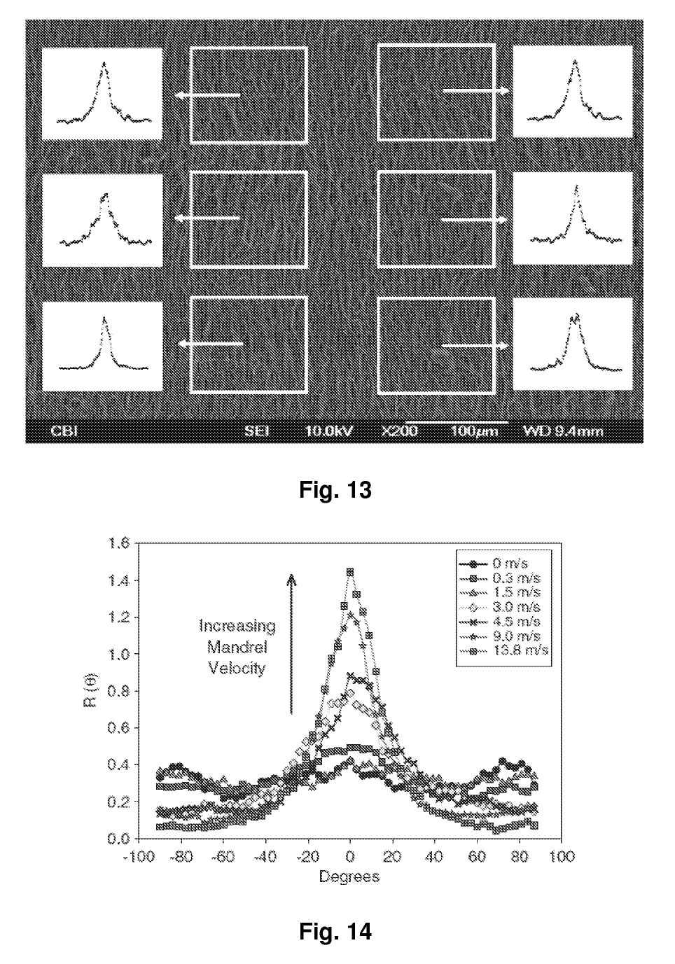

The custom image analysis software produced high fidelity tracking of the fibers in the SEM images (FIG. 12H). Using this method, it was determined that a high level of structural uniformity within each specimen existed (FIG. 13). By averaging the results from the six SEM images per specimen, the averaged data yielded results seen qualitatively in the SEM images. The random, 0.3 and 1.5 m/s scaffold data indicate little to no fiber alignment, whereas the data from scaffolds at or above 3.0 mls show increasing alignment with increasing mandrel velocity (FIG. 14).

2.7 Biaxial Mechanical Behavior

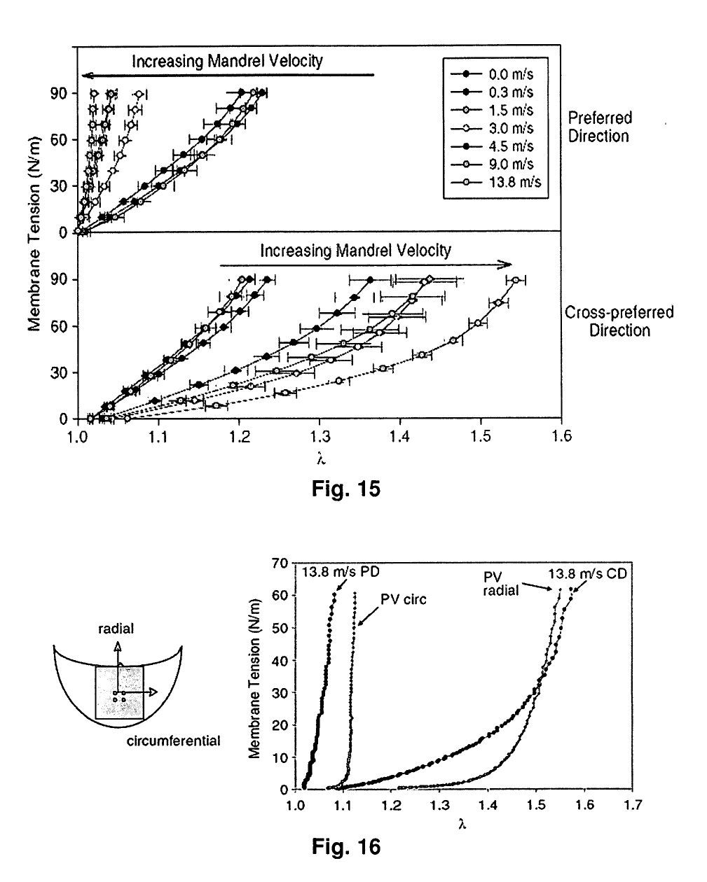

Under a state of planar equibiaxial mechanical stress (that is, where the two axial stress components are equal and the shear stress is zero), the random and low mandrel velocity specimens exhibit nearly the same mechanical response for both the preferred and cross-preferred fiber directions, with a maximum stretch λ=1.2 (FIG. 15). Above mandrel tangential velocities of 1.5 m/s, the mechanical response became substantially anisotropic, with increasing anisotropy with increasing mandrel velocity. The scaffold fabricated at 13.8 m/s mandrel velocity exhibited the highest amount of stretch in the cross-preferred direction and the lowest in the preferred direction. This was expected since that specimen had the most degree of alignment. Interestingly, all stress-strain curves exhibited a non-linear mechanical response reminiscent of soft tissues.

When the highest velocity group (13.8 m/s) was compared with planar biaxial mechanical data from the native pulmonary heart valve leaflet, a strong similarity in response was observed (FIG. 16), underscoring the ability of ES-PEUU scaffolds to simulate the anisotropic response of soft tissues.

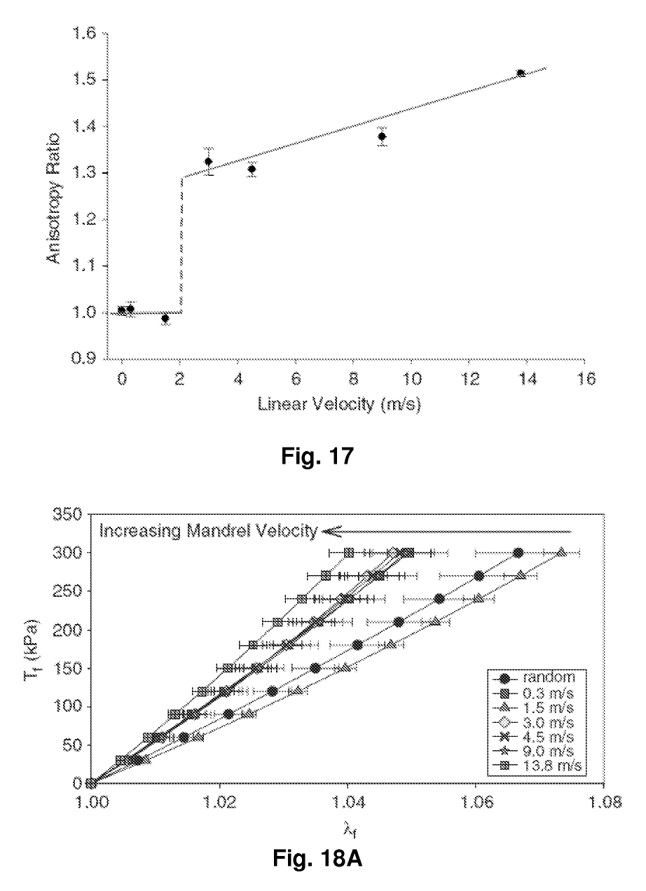

An interesting phenomenon was observed when the mechanical AR was compared to the mandrel tangential velocity (FIG. 17). No change from isotropy (AR=1) occurred at velocities less than ˜2 m/s. At tangential velocities greater than 2 m/s, the AR increased abruptly to ˜1.3, followed by a steady monotonic increase to 1.5 at 14 m/s. These results indicate highly controllable ranges of mechanical anisotropy by adjusting the rotation velocity.

2.8 Scaffold Structural Constitutive Model

Initial fits were done to the entire four-parameter model to determine whether Eq. (4) was over-parameterized. Results indicated the need for two parameters (A, B) for the random specimen (since R(θ)=1/π, so that d=1 and c is not used), three parameters (A, B, c) for 2.0 m/s or higher specimens, and four parameters for the 0.3 and 1.5 m/s specimens. The fit of the model to the data was good despite the complexity of the mechanical response over the broad range of biaxial loading states. The two-, three-, and four-parameter models fit the biaxial data quite well, with r²=0.93 or greater (Table 2) and the majority of fits being r²=0.97 or greater.

TABLE 2
Model fit parameters for Eq. (4) and porosity and crystallinity*

| Velocity | | | | | | | Porosity | Crystallinity |
|---|---|---|---|---|---|---|---|---|
| (m/s) | A (kPa) | B | c | d | r² PD | r² XD | (%) | (%) |
| 0.0 | 41022 ± 10544 | 0.220 ± 0.110 | n/a | n/a | 0.985 ± 0.0009 | 0.991 ± 0.0016 | 82 | 37 |
| 0.3 | 86250 ± 15770 | 0.079 ± 0.013 | 2016 ± 741 | 0.540 ± 0.152 | 0.976 ± 0.0067 | 0.986 ± 0.0026 | — | — |
| 1.5 | 7861 ± 5094 | 1.33 ± 0.34 | 5936 ± 1875 | 0.665 ± 0.160 | 0.970 ± 0.0104 | 0.979 ± 0.0068 | 78 | 58 |
| 3.0 | 1423 ± 231 | 4.67 ± 1.07 | 22.6 ± 7.12 | n/a | 0.988 ± 0.0014 | 0.983 ± 0.0016 | — | — |
| 4.5 | 7800 ± 3839 | 2.05 ± 0.58 | 20.7 ± 1.28 | n/a | 0.985 ± 0.0037 | 0.992 ± 0.0021 | 76 | 72 |
| 9.0 | 3400 ± 1132 | 2.16 ± 0.29 | 16.06 ± 1.37 | n/a | 0.983 ± 0.0030 | 0.991 ± 0.0015 | — | — |
| 13.8 | 3020 ± 670 | 2.75 ± 0.45 | 6.75 ± 0.47 | n/a | 0.931 ± 0.0126 | 0.935 ± 0.0229 | 72 | 100 |

*Porosity and crystallinity measures were only done for the samples indicted in the table. Crystallinity measure for the random, 1.5 and 4.5 m/s specimens are percentages with respect to the 13.8 m/s specimen. Thus, the 4.5 m/s sample is 72% as crystalline as the 13.8 m/s sample.

Effective fiber stress-strain results indicated a near linear fiber stress-strain response, with a monotonic increase in fiber stiffness with increasing mandrel velocity (FIG. 18A). This result is consistent with uniaxial mechanical response of PEUU. The predicted R(θ) also demonstrated a monotonic increase in fiber alignment with increasing mandrel velocity (FIG. 18B), which compared favorably with actual measured orientations (FIG. 11). It should be noted that the model tended to predict a slightly higher degree of orientation than was actually found from the SEM images. This was attributed to the fact that the model utilized overall fiber direction, whereas the SEM image analysis also accounted for fiber tortuosity, which resulted in a lower degree of alignment. Crystallinity measurements made on samples from 1.5, 4.5, and 13.8 m/s revealed that crystallinity increased with mandrel speed (Table 2). Since higher crystallinity is generally associated with higher chain (or fiber) moduli, the structural model predictions for the effective fiber properties support this conclusion (FIG. 18A).

2.9 Fiber Alignment

In certain embodiments the electrospinning process has been integrated with a rotating mandrel, with varying quantities of rotational speed. Also, newly described herein is biaxial testing, which is much more physiologically relevant especially for soft tissue constructs; and a structural model that provides feedback for future design of scaffolds. Induced fiber orientation of the scaffolds can be seen above the 2 m/s tangential velocity (FIGS. 12A-12H and 17). The 2 m/s speed appears to be a speed at or above which the fiber alignment changes for the electrospinning setup described herein. Scaffolds developed below this speed show little to no fiber alignment. The stress/stretch curves for these specimens are very similar, indicating that at a mandrel velocity of ≦2 m/s very little alignment takes place, if at all, and the scaffolds elicit mechanical responses expected of an isotropic fiber network. This result suggests that at tangential velocities ≦2 m/s, additional factors come into play that inhibit fiber orientation, and that increased orientation is not a simple function of mandrel rotation speed.

In the electrospinning process, the fiber is first a straight jet that approaches the target but then at some point becomes curved and more complicated. This curved path is due to an electrically driven bending instability of the charged jet. The trajectory of a typical segment moves both out and in towards the direction of the applied electric field between the tip and collector. The segment is also influenced from distant parts of the jet. The curved segment is bent and elongated by self-repulsion of electrical charges within that segment. In the case of a moving mandrel, the surface velocity of the mandrel has to exceed the fiber delivery rate in order for mandrel rotations to induce fiber orientation. Using a feeding rate of the 5% PEUU solution at 1 mL/h through a 1.19 mm ID capillary, the velocity of the feed solution at the nozzle is 9.4×10⁻⁶ m/s. Assuming a single fabricated fiber being delivered at 0.05 mL/h (5% of 1 mL/h for 5% PEUU in HFIP), the fiber diameter would have to be equal to 941 nm so that the velocity of the fiber would equal 2 m/s. Thus, it appears there are 3-5 fibers depositing concurrently to result in a 2 m/s velocity and isotropic fiber networks for tangential velocities below the 2 m/s threshold were the result of a solution feed rate in excess of this 2 m/s threshold.

Matsuda et al. [Matsuda T., et al., “Mechanoactive scaffold design of small diameter artificial graft made of electrospun segmented polyurethane fabrics”, J. Biomed. Mater. Res. A 2005; 72(1):117-124] recently investigated the effects of rotational speed on the anisotropic behavior of hollow tubular scaffolds. The scaffolds were electrospun onto a 3 mm diameter steel cylinder rotating at 150 rpm or 3400 rpm, which was also capable of transverse motion. Both speeds exhibited very little anisotropy, most likely attributable to the insufficient rotational speed. With a 3-mm diameter tube and a rotational speed of 3400 rpm, the linear velocity on the outside of the tube is only 0.53 m/s. Based on our results, a linear velocity of ˜0.5 m/s would yield a scaffold that exhibits an isotropic mechanical response with no preferred fiber orientation (FIG. 17). Thus, actual realized linear velocity, not revolution rate, in relation to estimated fiber delivery rate is a better predictor of fiber alignment and anisotropy.

In tissue engineering, the polymer scaffold is often used to temporarily provide the biomechanical structural characteristics for the replacement “tissue” until sufficient extracellular matrix is produced, which will ultimately provide the structural and mechanical integrity for the replacement “tissue.” Thus, it can be useful to know the structure and mechanical properties of the native tissue being replaced or strengthened through the addition of new tissue. Cells rely upon mechanical stimuli for feedback on replication, with part of this feedback coming in the form of the supporting matrix. There is a need for anisotropic mechanical properties to facilitate physiological function, as well as large strains to promote an aligned network of collagen and elastin fibers and overall tissue growth. Xu et al. showed that, by utilizing the electrospinning technique, an aligned fiber network could induce cell alignment in vessel tissue engineering [Xu C. Y., Inai R., Kotaki M., Ramakrishna S., “Aligned biodegradable nanofibrous structure: a potential for blood vessel engineering”, Biomaterials 2004 (25) 877-86.]. Riboldi et al. developed electrospun, biodegradable poly (ester) urethane scaffolds that could potentially serve as scaffolds for tissue engineering of skeletal muscle, with mechanical testing done to demonstrate tensile strength [Riboldi S. A., “Electrospun degradable polyesterurethane membranes: potential scaffolds for skeletal muscle tissue engineering”, Biomaterials 20005; 26(22):4606-4615]. However, neither of these studies showed that the scaffolds could produce mechanical responses needed for the application in question. A scaffold is provided herein that yields a mechanical response quite similar to the native pulmonary valve. This property is quite desirable, especially in bioreactor studies where tissues are mechanically trained to promote extracellular matrix deposition and strength.

### Example 3

### Cyclic Flexural Stimulation of Tissue Engineering Pulmonary Valve Biomaterials

3.1 Cyclic Flexure of PGA/PLLA Scaffolds

Cyclic flexure is a major mode of deformation experienced by native and TEPV leaflets during the opening and closing phases of normal valve function. To elucidate the independent role of cyclic flexure in TEPV development, a sensitive three-point bending test was used to evaluate the mechanical stability of candidate TEPV scaffold materials under cyclic flexure. While poly-4-hydroxybutrate (P4HB) dip-coated non-woven PGA displayed a predictable decline in effective stiffness (E) with either static of cyclic flexural incubation (data not shown), P4HB dip-coated 50:50 PGA/PLLA exhibited a sharp drop in E to nearly baseline levels upon flexure, obviating the reinforcing effect of the P4HB dip-coating.

Based on these results, the independent role of cyclic flexure in the in-vitro development of smooth muscle cell (SMC) seeded TEPV was investigated. SMC's isolated from ovine carotid artery were expanded in-vitro and dynamically seeded onto rectangular strips of the non-woven 50:50 PGA/PLLA scaffold. Following 30 hours seeding and 4 days static incubation, SMC-seeded scaffolds (n=6) were transferred to the bioreactor and subjected to unidirectional cyclic flexure at a physiological frequency (1 Hz) and amplitude for 3 weeks. SMC-seeded scaffolds maintained under static conditions (n=6) and unseeded scaffolds served as controls. The virgin non-woven 50:50 PGA/PLLA scaffold was mechanically stable over the 3 weeks duration of the experiment. Several-fold increases in E with the accumulation of relative low concentrations of collagen were observed independent of concurrent changes in scaffold mechanical properties. Thus, in addition to the several independent effects of cyclic flexure on early in-vitro TEPV development, this example demonstrates the phenomenon of collagenous reinforcement in an engineered tissue based on a synthetic scaffold.

3.2 Cyclic Flexure and Laminar Flow Synergistically Accelerate Mesenchymal Stem Cell Mediated Engineered Heart Valve Tissue Formation.

Pluripotent bone marrow-derived mesenchymal stem cells (BMSC) can be isolated relatively non-invasively, and thus represent a potential cell source for tissue engineered heart valves (TEHV). Ovine BMSC-seeded TEHV previously functioned for at least 8 months in the pulmonary outflow tract of sheep. Toward optimizing mechanical conditioning regimens, independent and coupled effects of cyclic flexure and laminar flow on BMSC-mediated tissue formation is recognized herein. Ovine BMSC were seeded onto nonwoven 50:50 blend PGA/PLLA scaffolds and maintained in static culture for 4 days prior to loading in our flex-stretch-flow (FSF) bioreactor, BMSC-seeded scaffolds were incubated under static (n=12), cyclic flexure (1 Hz, Δk=0.554 mm−1; n=12), laminar flow (π=1.1505 dyne/cm2; n=12) and combined flex-flow (n=12) conditions for (n=6) and 3 (n=6) weeks and then characterized by effective stiffness (E) testing, DNA and extracellular matrix assays, histology, immunohistochemistry, and scanning electron microscopy (SEM). Results indicated by 3 weeks, the flex-flow group exhibited dramatically accelerated tissue formation compared with all other groups, including a 74% increase in collagen concentration (844±278 versus 483±55 μg/g wet weight, respectively; p<0.05). Thus, for this cell/scaffold combination cyclic flexure and laminar flow synergistically (that is, more than the simple sum of their respective contributions) accelerated tissue formation.

### Example 4

### Electrospun Tubular Constructs for Blood Vessel Tissue Engineering

One consideration for the development of blood vessel replacements is accurate replication of the original vessel compliance. Compliance mismatch is a complex phenomenon because it involves the host artery, anastomosis, and the graft itself. Blood flow can be traumatized causing turbulence and low shear stress that favors platelet deposition. These complications can further lead to myointimal hyperplasia and graft failure. Therefore, in developing a blood vessel replacement, it is useful to not only create a non-thrombogenic luminal surface but to also closely replicate the elastic properties of the vessel wall.

This example describes one embodiment of a method to produce a highly cellularized blood vessel construct that is capable of also providing substantial elastomeric mechanical support. The method involves a micro-integrated approach wherein a meshwork of submicron elastomeric fibers is built into a vessel wall with or without the cellular placement process. Cellularity can be developed through in vitro culture methods or in vivo.

A method also is provided herein to luminally surface seed small diameter electrospun polyurethane conduits that may be used for aorta replacements in vivo. Also provided is electrospinning technology to incorporate cells during scaffold fabrication to better encourage tissue development. As discussed herein, the constructs were characterized for their cellularity and mechanical properties.

4.1 Tubular Electrospinning and Scaffold Characterization

Poly(ester urethane)urea was synthesized from poly(ε-caprolactone)diol and 1,4-diisocyanatobutane with putrescine chain extension as described herein. PEUU was dissolved at 6 wt % in hexafluoroisopropanol and electrospun. Electrospinning conditions included a solution volumetric flowrate of 1.0 mL/hr, a distance between nozzle and target of 13.5 cm, and voltages of +12 kV to the nozzle and −3 kV to the target. The target used for fabrication of small diameter tubes for implantation was a Type 316 stainless steel mandrel of 1.3 mm diameter that was rotating at 250 rpm.

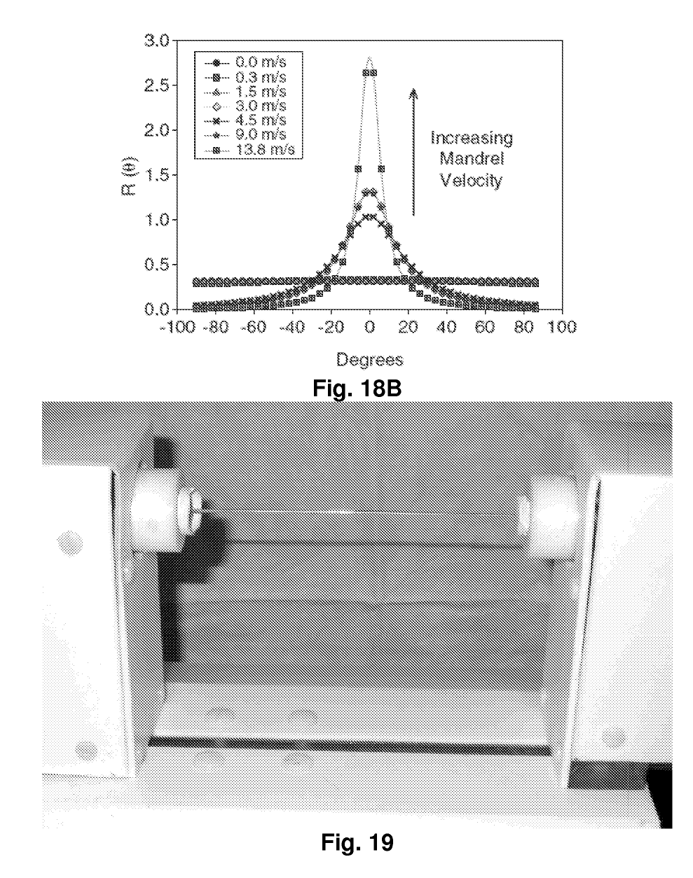

An image of this custom-designed and constructed target is displayed in FIG. 19. This mandrel was also translating along its axis 8 cm on a linear stage at a speed of approximately 8 cm/s to produce a more uniform conduit thickness. Samples were electrospun for 15 min to produce porous tubular constructs with wall thicknesses on the order of 150 to 200 μm. For endothelialization studies a 4.7 mm stainless mandrel was instead utilized with the same process conditions.

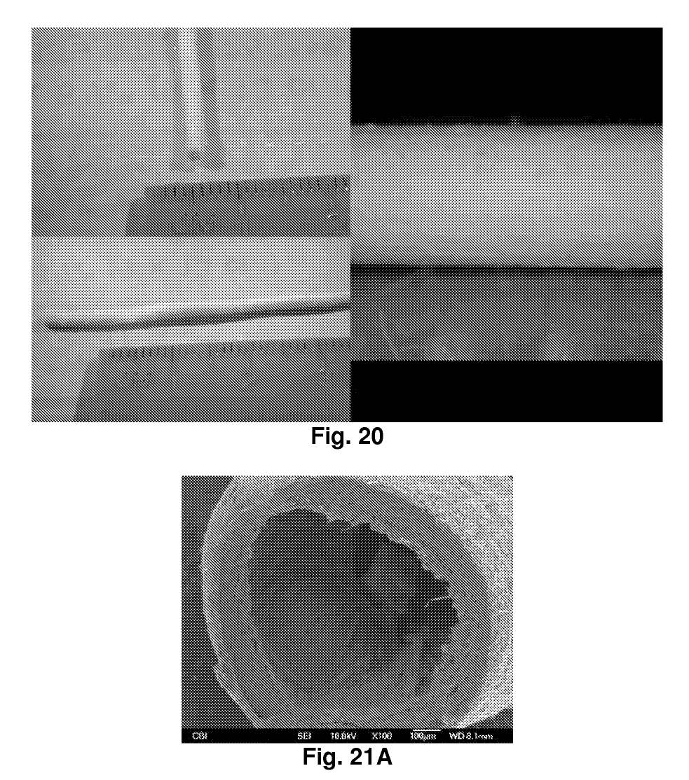

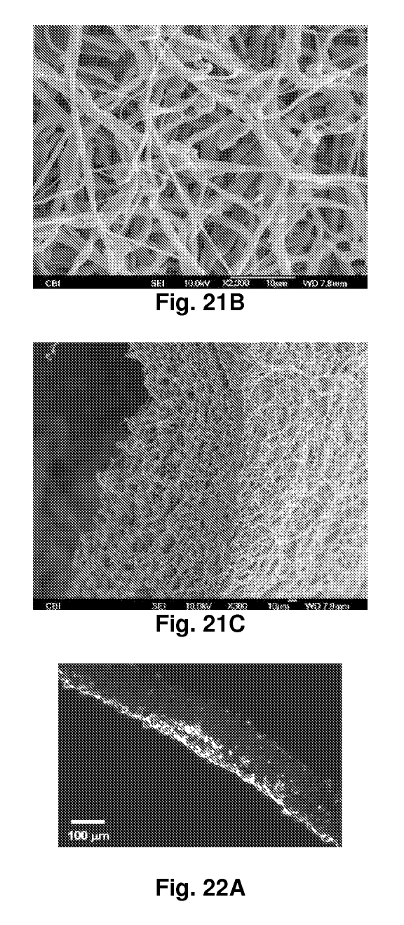

PEUU at 6 wt % in HFIP was electrospun onto a negatively charged rotating mandrel at 250 rpm to produce a tubular construct. FIG. 20 demonstrates the gross appearance of the conduit. The electrospun tubes possessed 1.3 mm inner diameters, lengths up to 8 cm and wall thicknesses of 150-200 μm. The fibrous structures of the scaffold tubes are shown by SEM in FIGS. 21A-21C. One can observe fiber sizes approximately in the range of 1000 m. In addition, these constructs were suturable and retained their lumens.

After fabrication, the mandrel was dipped in 70% ethanol in order to more easily remove it from the steel mandrel. The conduit was then rinsed in deionized water multiple times, blotted dry and then dried under vacuum at room temperature 24 to 48 h. Conduits were then examined for their gross structure with a dissecting microscope or their fibrous morphologies with scanning electron microscopy. In order to view an uninterrupted fibrous cross-section, samples were dipped in liquid N₂ for 1 min and then fractured before sputter-coating for SEM.

4.2 Surface Seeding of Conduit Lumen

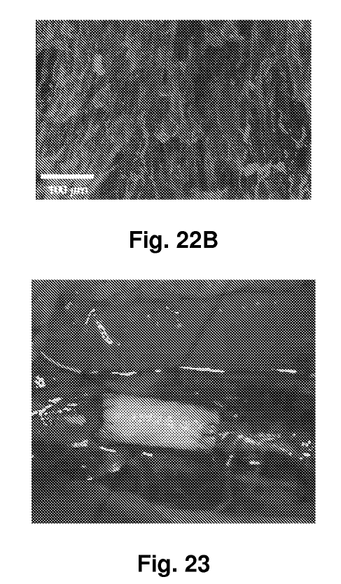

PEUU conduits (4.7 mm) were positioned inside a custom designed rotational vacuum seeding device and seeded with 20×10⁶ muscle derived stem cells (MDSCs). More specifically, the electrospun conduit was placed on metal stubs and a light vacuum was applied to the exterior of the conduit. Subcultured MSDCs were then perfused through the lumen of the conduit and forced into the fibrous lumen side wall of the tube by vacuum. Constructs were cultured under static conditions in Petri dishes for 24 h. After 24 h of static culture, cells were viable, adhered to the lumen and formed a monolayer. Samples were then fixed in 2% paraformaldehyde before permeabilization with 0.1% triton-x100 and staining with DAPI or draq 5 nuclear stain and rhodamine phallodin for f-actin and imaged with fluorescence microscopy. An image depicting the cells lining the construct interior is shown in FIGS. 22A and 22B. This image was a fluorescent micrograph depicting the cell nuclei and f actin staining.

4.3 In Vivo Implantation as a Rat Aorta Replacement

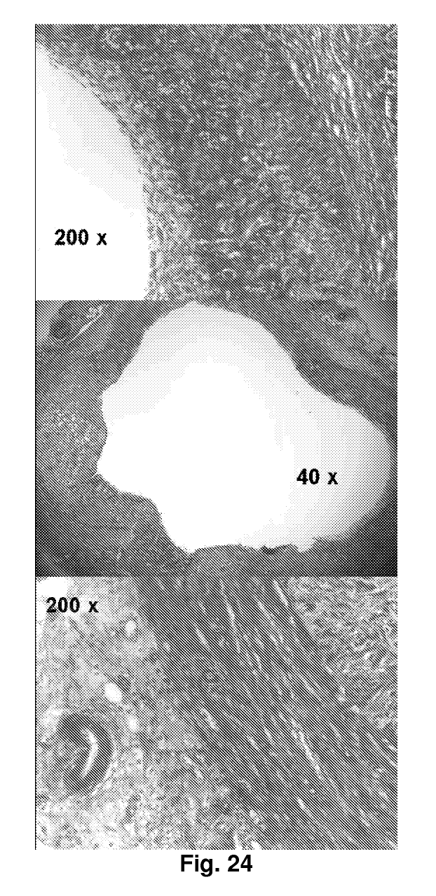

Porous 1.3 mm inner diameter tubular electrospun scaffolds were implanted as interposition grafts in the abdominal aorta of rats. Constructs were suturable and easily retained their lumens in vivo. Lewis female rats weighing 250-300 g were anesthetized with 1% isofluorane and 2.5 2.5 mg/100 g ketamine. A mid-abdominal incision was performed and the retroperitoneal cavity exposed. The descending aorta below renal level was dissected, clamped proximally and distally sectioned to make a 1 cm gap. The electrospun conduit was then implanted in an end-to-end manner using prolene 10.0 sutures. Intravenous heparin was administered before clamping with 200 Units/kg. An image of the graft immediately after implantation is shown in FIG. 23. The abdominal wall was closed in two layers with Vycril 2.0 sutures. Rats were able to recover from the surgeries with limb function. Rats were sacrificed at 2 wks and sample explants fixed in 10% neutral buffered formalin at room temperature. At 2 wks after implantation, grafts remained patent and functional. Samples were then embedded in paraffin and sectioned before staining with Hematoxylin and Eosin or Masson's Trichrome. Hematoxylin and eosin staining demonstrated external capsule formation around the explanted grafts. Masson's Trichrome staining indicated the capsule was composed of aligned collagen together with the presence of newly developed capillary vessels. Cell and tissue in-growth was observed throughout the constructs with the presence of collagen development. Cells were also demonstrated to have formed a monolayer in locations around the construct lumens. Images representative of histological examination of the 2 wk explants are displayed in FIG. 24.

### Example 5

### SMC Microintegrated Polyurethane Conduits

Whereas the previous example provided in vivo approach, a biodegradable and cytocompatible, elastomeric poly(ester urethane)urea was electro spun into small diameter tubes appropriate for implantation in a rat model.

Like the previous example, this example provides methods for fabricating a highly cellularized blood vessel construct that also provides substantial elastomeric mechanical support. However, the previous model was an in vivo approach in a biodegradable and cytocompatible, elastomeric poly(ester urethane)urea was electro spun into small diameter tubes appropriate for implantation in a rat model. This example provides an in vitro approach, wherein SMCs were seeded into electrospun nanofibers concurrently with scaffold fabrication using a microintegration technique.

5.1 Conduit Microintegration Technique

Vascular smooth muscle cells (SMCs) isolated from rat aortas were expanded on tissue culture polystyrene (TCPS) culture plates under Dulbecco's Modified Eagle Medium (DMEM) supplemented with 10% fetal bovine serum and 1% penicillin-streptomycin. Microintegration was performed similar to described previously with some modifications to allow for a smaller diameter electrospraying/electro spinning mandrel.

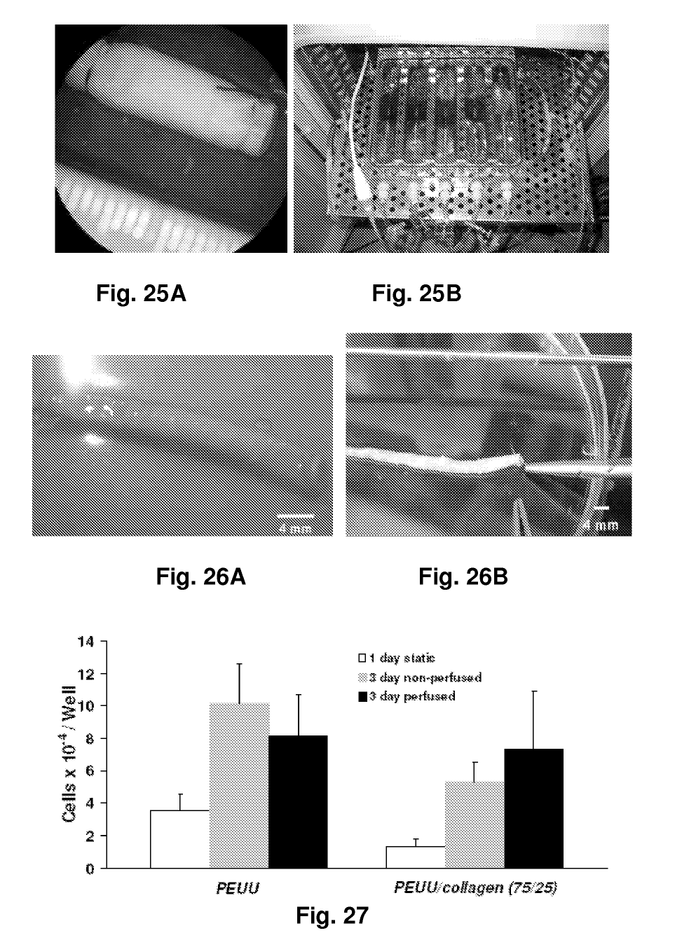

7.5×10⁶ SMCs/mL were subcultured in medium and fed at 0.1 mL/min into a sterile Type 316 stainless steel capillary charged at 8.5 kV and located 4.5 cm from the target. 6 wt % PEUU or 6 wt % PEUU/collagen (75/25) in HFIP was fed at 1.5 mL/min into a capillary charged at 12 kV and located 23 cm from the target. The target consisted of a sterile stainless steel mandrel (4.7 mm diameter) charged at −3 kV and rotating at 250 rpm while translating 8-cm along its axis at 1.6 mm/s. A fabrication time of 30 min was used to produce each microintegrated conduit. After fabrication the conduit and mandrel were gently placed with aseptic technique into a roller bottle and cultured statically for 16 h. After 16 h, samples were gently removed from the mandrel for culture. Samples were then cut into 15 mm lengths and sutured to metal stubs and perfused media with pulsatile flow for 3 days. Images depicting the perfusion sample and reactor are shown in FIGS. 25A and 25B.

5.2 Conduit Characterization

At timepoints of 1 day and 4 days after fabrication, samples were characterized. The MTT mitochondrial assay was used to measure cell viability. For histological investigation, samples were fixed in 10% neutral buffered formalin at room temperature. Samples were then embedded in paraffin, sectioned and stained with hematoxylin and eosin. Samples were analyzed for their biomechanical properties immediately after fabrication. Properties measured included ring strength, dynamic compliance, and burst pressure. In order to measure ring strength, stainless steel staples were inserted into 5 mm long tubular sections and then into the grips of a uniaxial tensile tester (A TS). Using a 10 lb load cell and a displacement rate of 10.05 mm/min samples were strained until break.

For dynamic compliance and burst strength, 15 mm long tubular samples were mounted in a flow loop driven by a centrifugal pump (Biomedicus) and submerged in PBS at 37° C. The pressure was monitored and recorded at 30 Hz using a standard in-line strain-gage pressure transducer and a PC acquisition board. The vessel construct was perfused with a pulsatile flow (110-70 mmHg, 1.2 Hz) and the dynamic compliance, C, was measured by recording the external diameter of the sample with a He—Ne laser micrometer (Lasermike). Compliance was calculated as:

$$C = \frac{( {D}_{max} - {D}_{min} )}{{D}_{min} ( {P}_{max} - {P}_{min} )}$$

for each pulse (D=maximum or minimum diameter, P=maximum or minimum pressure). A porcine mammary artery was used as a control for comparison with microintegrated PEUU in compliance studies. For measuring burst pressure, the sample outlet was sealed and flow was increased until tube rupture. The maximum pressure before rupture was taken as the burst pressure.
5.3 Scaffold Structure for Microintegration

In order to extend the technology of cellular microintegration to small diameter tubes, a 4.7 mm diameter stainless steel mandrel was used in the place of the previously employed 19 mm diameter mandrel for sheet microintegration. In order to microintegrate highly cellular and defect free tubular constructs, it was useful to slightly decrease electrospraying distance 0.5 cm and lower the mandrel negative charge from −10 kV to −3 kV from previous methods. During fabrication, PEUU appeared pink and glistening on the mandrel indicative of uniform cellular electrospray. After removal from the mandrel, samples of either PEUU or PEUU/collagen (75/25) were found to be mechanically robust in that they were suturable and could retain their lumens after compression. Images depicting the suturability and gross appearance of SMC micro integrated PEUU conduits are illustrated in FIGS. 26A and 26B.

5.4 Cell Growth and Histology

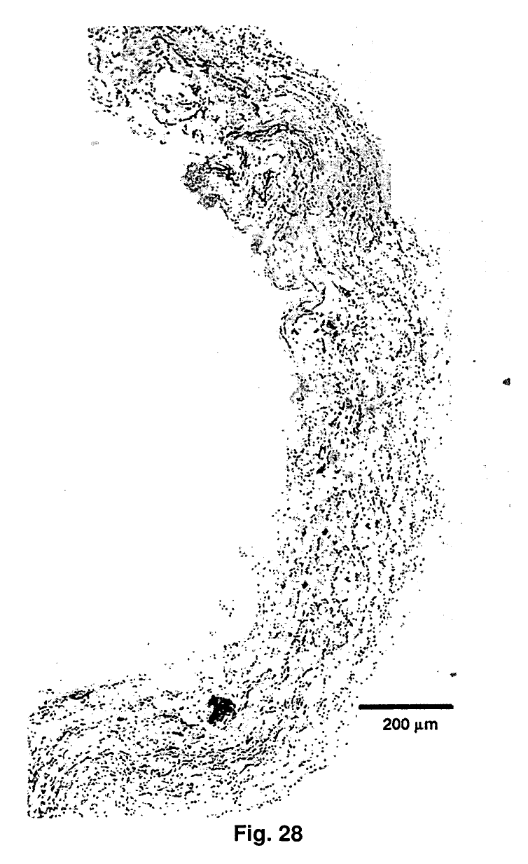

Cell placement and viability in the SMC micro integrated constructs was investigated initially and again after 4 days of static or perfusion culture. After perfusion, samples were gently removed from the stubs and then sectioned into representative slices for MTT and histology. MTT results indicated viable cells 1 day after fabrication. Furthermore, cells remained viable at day 4 with either static or perfusion culture with cell number values reported slightly higher for perfusion culture. MTT data are summarized in FIG. 27. Samples were fixed and stained with hematoxylin and eosin staining. A representative H&E stain of uniform initial cell integration within the tubular construct is shown in FIG. 28. This half-tube image consists of multiple images taken ITom the tube periphery grouped together to create a representative image.

5.5 Mechanical Properties

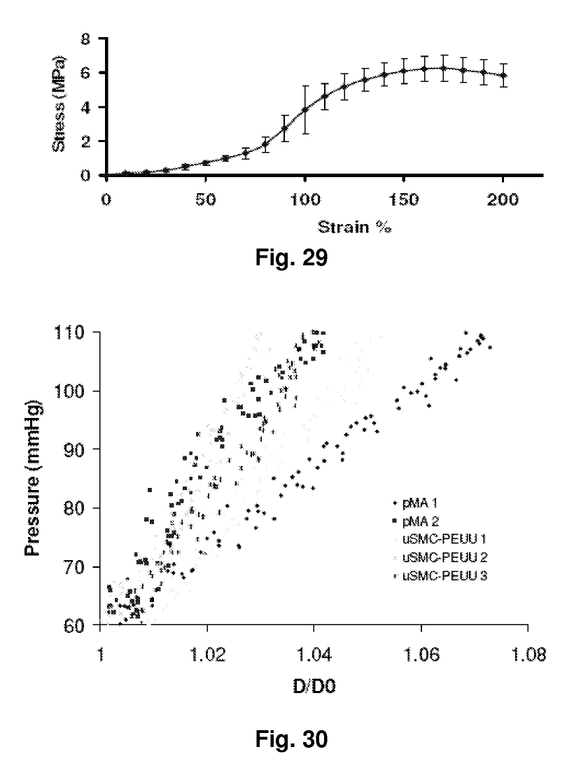

Ring strength, burst pressure, and suture retention strength were assessed in the micro integrated constructs after fabrication. The stress strain response from subjecting a small tube section to uniaxial tensile testing is displayed in FIG. 29. These rings were mechanically robust and flexible with maximum stress and strain values of 6.3 MPa and 170% respectively. The ring samples did not break cleanly in each case and seemed to pull apart or delaminate past the ultimate stress value. In order to calculate the dynamic compliance of the microintegrated constructs, samples were exposed to pulsatile flow and the pressure/diameter relationship was evaluated. This relationship was compared with a porcine mammary artery (pMA) exposed to the same pulsatile flow. As seen in FIG. 30, the mechanical response of both the pMA and microintegrated PEUU was very similar with values falling for both samples falling between one another. Compliance values were 1.02±0.33×10⁻³ mmHg⁻¹ for pMA and 0.71±0.13×10⁻³ mmHg⁻¹ for SMC microintegrated PEUU. Burst pressure values for all samples were greater than 1500 mmHg. The burst pressure values were approximations due to the porous nature of the microintegrated tubes.

This method produced highly cellularized elastomeric scaffolds. Cells were viable after fabrication and proliferated under perfusion culture. In order to extend this technology to micro integrate cells into small diameter tubular constructs as a blood vessel prototype, it was advantageous to modify some process variables. For example, in order to target and electro spray cells onto the smaller diameter mandrel it was useful to decrease the distance between electro spray nozzle and mandrel. Also, it was useful to avoid a large negative bias on the mandrel. Using a high negative charge to the rotating mandrel target resulted in polymer protrusion defects, or “spikes,” in the tube which could disrupt conduit integrity and cell viability. Therefore, it was useful to decrease mandrel charge to result in homogenously cellular and fibrous tubular conduits. These constructs were then cultured under a perfusion bioreactor to encourage better exchange of nutrients, waste, and oxygen to the cells in the tube interior. H&E and MTT results indicated viable cells present within the constructs after fabrication and perfusion culture.

Having described this invention above, it will be understood to those of ordinary skill in the art that the same can be performed within a wide and equivalent range of conditions, formulations and other parameters without affecting the scope of the invention or any embodiment thereof.

We claim:

1. A prosthetic cardiovascular valve leaflet comprising a biodegradable elastomeric scaffold having anisotropic mechanical properties and comprising cells integrated into the scaffolding.

2. The prosthetic cardiovascular valve leaflet of claim 1, wherein the biodegradable elastomeric scaffold is a non-woven mesh having a plurality of pores.

3. The prosthetic cardiovascular valve leaflet of claim 2, wherein the non-woven mesh is formed by electrospraying.

4. The prosthetic cardiovascular valve leaflet of claim 3, wherein the non-woven mesh is formed by electrospinning.

5. The prosthetic cardiovascular valve leaflet of claim 2, wherein cells are microintegrated into the pores of the non-woven mesh.

6. The prosthetic cardiovascular valve leaflet of claim 5, wherein the cells are microintegrated by electrospraying.

7. The prosthetic cardiovascular valve leaflet of claim 2, wherein the cells that are microintegrated into the pores of the non-woven mesh are chosen from one or more of stem cells, precursor cells, smooth muscle cells, skeletal myoblasts, myocardial cells, endothelial cells, endothelial progenitor cells, bone-marrow derived mesenchymal cells and genetically modified cells.

8. The prosthetic cardiovascular valve leaflet of claim 1, incorporated into a prosthetic cardiovascular valve.

9. The prosthetic cardiovascular valve leaflet of claim 1, wherein the biodegradable elastomeric scaffold further comprises a therapeutic agent and/or a growth factor.

10. The prosthetic cardiovascular valve leaflet of claim 9, wherein the therapeutic agent is an antiinflammatory agent chosen from one or more of salicylic acid, indomethacin, sodium indomethacin trihydrate, salicylamide, naproxen, colchicine, fenoprofen, sulindac, diflunisal, diclofenac, indoprofen sodium salicylamide, antiinflammatory cytokines, antiinflammatory proteins, and steroidal antiinflammatory agents.

11. The prosthetic cardiovascular valve leaflet of claim 9, wherein the therapeutic agent is an anticlotting factor.

12. The prosthetic cardiovascular valve leaflet of claim 11, wherein the anticlotting factor is heparin.

13. The prosthetic cardiovascular valve leaflet of claim 9, wherein the growth factor is chosen from one or more of an angiogenic or neurotrophic factor, basic fibroblast growth factor (bFGF), acidic fibroblast growth factor (aFGF), vascular endothelial growth factor (VEGF), hepatocyte growth factor (HGF), insulin-like growth factors (IGF), transforming growth factor-beta pleiotrophin protein, and midkine protein.

14. The prosthetic cardiovascular valve leaflet of claim 1, wherein the prosthetic cardiovascular valveleaflet is adapted to replace a cardiovascular valve leaflet of one of a venous valve, a mitral valve, an aortic valve, a pulmonary valve, and a tricuspid valve.

15. The prosthetic cardiovascular valve leaflet of claim 1, wherein the prosthetic cardiovascular valve leaflet is adapted to replace a cardiovascular valve leaflet of one of a venous valve, a pulmonary valve, and a tricuspid valve.

16. The prosthetic cardiovascular valve leaflet of claim 1, wherein the prosthetic cardiovascular valve leaflet is adapted to replace a leaflet of a pulmonary valve.

17. A method of repairing a damaged pulmonary valve or venous valve in a patient, comprising implanting in the patient a prosthetic cardiovascular valve leaflet comprising a biodegradable elastomeric scaffold having anisotropic mechanical properties and comprising cells integrated into the scaffold or a prosthetic cardiovascular valve comprising the prosthetic cardiovascular valve leaflet

18. A prosthetic blood vessel comprising a tube, wherein the tube comprises a non-woven biodegradable elastomeric scaffold having a plurality of pores, and wherein cells are optionally microintegrated into the pores of the biodegradable elastomeric scaffold.

19. The prosthetic blood vessel of claim 18, wherein the cells are chosen from one or more of stem cells, precursor cells, smooth muscle cells, skeletal myoblasts, myocardial cells, endothelial cells, endothelial progenitor cells, bone-marrow derived mesenchymal cells and genetically modified cells.

20. A prosthetic vocal fold, wherein the prosthetic vocal fold comprises a biodegradable elastomeric scaffold, and wherein cells are optionally microintegrated into the biodegradable elastomeric scaffold.

21. The prosthetic vocal fold according to claim 20, wherein the cells are selected from the group consisting of stem cells, precursor cells, smooth muscle cells, skeletal myoblasts, myocardial cells, endothelial cells, endothelial progenitor cells, bone-marrow derived mesenchymal cells and genetically modified cells.
